(19) United States
(12) Patent Application Publication
(10) Pub. No.: US 2012/0020881 A1
(43) Pub. Date: Jan. 26, 2012

(54) Triaryl-sulphonium compounds, kit and methods for labeling positron emitting isotopes

(75) Inventors: Lutz Lehmann, Berlin (DE); Timo Stellfeld, Berlin (DE); Keith Graham, Berlin (DE); Jessica Becaud, Olten (CH); Linjing Mu, Lenzburg (CH)

(73) Assignee: BAYER SCHERING PHARMA AKTIENGESELLSCHAFT, Berlin (DE)

(21) Appl. No.: 13/139,145

(22) PCT Filed: Dec. 4, 2009
(86) PCT No.: PCT/EP2009/008667

§ 371 Date: Oct. 11, 2011

(30) Foreign Application Priority Data
Dec. 12, 2008 (EP) 08075942.6

(51) Int. Cl. A61K 51/08; C07D 277/82; C07K 7/06; C07H 21/02; C07K 5/103; C07D 401/12; C07C 381/12; C07D 271/113; C07D 451/02; C07D 401/06; C07D 211/32; C07D 471/04; A61K 38/08; A61K 38/07; A61K 31/7088; A61K 31/428; A61K 31/496; A61K 31/185; A61K 31/4245; A61K 31/4427; A61K 31/46; A61K 31/517; A61K 31/445; A61K 31/519; A61P 25/00; A61K 51/04

(52) U.S. Cl. 424/1.69; 424/1.89; 548/163; 530/328; 536/23.1; 530/330; 544/365; 424/1.73; 562/30; 548/144; 546/268.1; 546/125; 544/360; 544/285; 546/225; 544/282; 514/21.7; 514/21.9; 514/21.6; 514/17.7; 514/44 R; 514/367; 514/253.12; 514/578; 514/364; 514/210.18; 514/304; 514/266.22; 514/330; 514/259.41

(57) ABSTRACT

This invention relates to novel compounds suitable for labeling by positron emitting isotopes, such as ¹⁸F, ¹¹C, ¹³N and ¹⁵O, through appropriate labeling reagents, such as ¹⁸F reagents and methods of preparing such a compound, compositions comprising such compounds, kits comprising such compounds or compositions and uses of such compounds, compositions or kits for diagnostic imaging by positron emission tomography (PET).

### FIELD OF INVENTION

This invention relates to novel compounds suitable for labeling by positron emitting isotopes, such as ¹⁸F, ¹¹C, ¹³N and ¹⁵O, through appropriate labeling reagents, such as ¹⁸F reagents and methods of preparing such a compound, compositions comprising such compounds, kits comprising such compounds or compositions and uses of such compounds, compositions or kits for diagnostic imaging by positron emission tomography (PET).

### BACKGROUND

Molecular imaging has the potential to detect disease progression or therapeutic effectiveness earlier than most conventional methods in the fields of oncology, neurology and cardiology. Of the several promising molecular imaging technologies having been developed as optical imaging and MRI, PET is of particular interest for drug development because of its high sensitivity and ability to provide quantitative and kinetic data.

Positron emitting isotopes include carbon, nitrogen, and oxygen. These isotopes can replace their non-radioactive counterparts in target compounds to produce tracers that function biologically and are chemically identical to the original molecules for PET imaging. On the other hand, ¹⁸F is the most convenient labeling isotope due to its relatively long half life (109.6 min) which permits the preparation of diagnostic tracers and subsequent study of biochemical processes. In addition, its low β+ energy (635 keV) is also advantageous.

The aliphatic and aromatic ¹⁸F-fluorination reaction are of great importance for ¹⁸F-labeled radiopharmaceuticals which are used as in vivo imaging agents targeting and visualizing diseases, e.g. solid tumours or diseases of brain (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). A very important technical goal in using ¹⁸F-labelled radiopharmaceuticals is the quick preparation and administration of the radioactive compound due to the fact that the ¹⁸F isotopes have a short half-life of about only 110 minutes.

For the synthesis of [F-18] labeled compounds it is necessary to react the [F-18]-fluoride anion with a molecule comprising a leaving group.

Typical aliphatic leaving groups are tosylates, mesylates, triflates and bromides (review: Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50).

Suited aromatic leaving groups for the [F-18] radiofluorination are e.g. nitro, aryl iodonium salts (J. Label Comp Radiopharm (1995), 37, 120-122; J. Label Comp Radiopharm (1997), 40, 50-52); J. Label Comp Radiopharm (2004), 47, 429-441) and trimethyl ammonium salts (Int J Appl Radiat Isot, (1982), 33, 445-448; J. Label Comp Radiopharm (1989), 27, 823-833).

Various methods of radiofluorination have been published using different precursors or starting material for obtaining ¹⁸F-labeled peptides. Due to the smaller size of peptides, both higher target-to-background ratios and rapid blood clearance can often be achieved with radiolabeled peptides. Hence, short-lived positron emission tomography (PET) isotopes are potential candidates for labelling peptides. Among a number of positron-emitting nuclides, fluorine-18 appears to be the best candidate for labelling bioactive peptides by virtue of its favourable physical and nuclear characteristics. The major disadvantage of labelling peptides with ¹⁸F is the laborious and time-consuming preparation of the ¹⁸F labelling agents. Due to the complex nature of peptides and several functional groups associated with the primary structure, ¹⁸F-labeled peptides are not prepared by direct fluorination. Hence, difficulties associated with the preparation of ¹⁸F-labeled peptide were alleviated with the employment of prosthetic groups as shown below. Several such prosthetic groups have been proposed in the literature, including N-succinimidyl-4-[¹⁸F]fluorobenzoate, m-maleimido-N-(p-[¹⁸F]fluorobenzyl)-benzamide, N-(p-[¹⁸F]fluorophenyl) maleimide, and 4-[¹⁸F]fluorophenacylbromide. Almost all of the methodologies currently used today for the labeling of peptides and proteins with ¹⁸F utilize active esters of the fluorine labeled synthon.

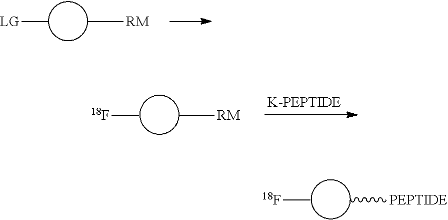

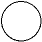

=aliphatic, aromatic or hetero-aromatic, alicyclic

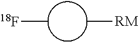

### =PROSTHETIC GROUP

RM=reactive moiety
LG=Leaving group that can be replaced by ¹⁸F
K=functional group for reaction with RM

Okarvi et al. (“Recent progress in fluorine-18 labeled peptide radiopharmaceuticals.” Eur. J. Nucl. Med., 2001 July; 28(7):929-38)) present a review of the recent developments in ¹⁸F-labeled biologically active peptides used in PET.

Xianzhong Zhang et al. (“¹⁸F-labeled bombesin analogs for targeting GRP receptor-expressing prostate cancer.” J. Nucl. Med., 47(3):492-501 (2006)) relate to the 2-step method detailed above. [Lys3]Bombesin ([Lys3]BBN) and aminocaproic acid-bombesin(7-14) (Aca-BBN(7-14)) were labeled with ¹⁸F by coupling the Lys3 amino group and Aca amino group, respectively, with N-succinimidyl-4-¹⁸F-fluorobenzoate (¹⁸F-SFB) under slightly basic condition (pH 8.5). Unfortunately, the obtained ¹⁸F-FB-[Lys3]BBN is metabolically relatively unstable having for result to reduce the extent of use of the ¹⁸F-FB-[Lys3]BBN for reliable imaging of tumor.

Thorsten Poethko et al. (“Two-step methodology for high-yield routine radiohalogenation of peptides: ¹⁸F-labeled RGD and octreotide analogs.” J. Nucl. Med., 2004 May; 45(5):892-902) relate to a 2-step method for labelling RGD and octreotide analogs. The method discloses the steps of radiosynthesis of the ¹⁸F-labeled aldehyde or ketone and the chemoselective ligation of the ¹⁸F-labeled aldehyde or ketone to the aminooxy functionalized peptide.

Thorsten Poethko et al. (“First ¹⁸F-labeled tracer suitable for routine clinical imaging of somatostatin receptor-expressing tumors using positron emission tomography.” Clin. Cancer Res., 2004 Jun. 1; 10(11):3593-606) apply the 2-step method for the synthesis of ¹⁸F-labeled carbohydrated Tyr(3)-octreotate (TOCA) analogs with optimized pharmacokinetics suitable for clinical routine somatostatin-receptor (sst) imaging.

WO 2003/080544 A1 and WO 2004/080492 A1 relate to radiofluorination methods of bioactive peptides for diagnostics imaging using the 2-step method shown above.

The most crucial aspect in the successful treatment of any cancer is early detection. Likewise, it is crucial to properly diagnose the tumor and metastasis.

Routine application of ¹⁸F-labeled peptides for quantitative in vivo receptor imaging of receptor-expressing tissues and quantification of receptor status using PET is limited by the lack of appropriate radiofluorination methods for routine large-scale synthesis of ¹⁸F-labeled peptides. There is a clear need for radiofluorination method that can be conducted rapidly without loss of receptor affinity by the peptide and leading to a positive imaging (with reduced background), wherein the radiotracer is stable and shows enhanced clearance properties

The conversions of mono-(mainly para-) substituted trimethylammonium benzene derivatives (1) to substituted [¹⁸F]-fluoro-benzene derivatives (2) which may serve as radiopharmaceutical itself or as prosthetic group for the F-18 labeling of small and large molecules have been reported in the literature (Irie et al. 1982, Fluorine Chem., 27, (1985), 117-191; Haka et al. 1989) (see Scheme 1).

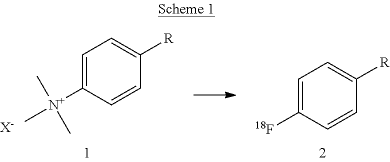

The drawback of the trimethyl ammonium based leaving group is the formation of possible side products (like [¹⁸F]methyl fluoride) (review Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, page 27)

Sulphonium derivatives based on three aryl or heteroaryl moieties are described in literature. For example it has been reported on para-substituted sulphonium derivative la (J. Mater. Chem.; 17; 7; 2007; 632-641) or 1b (J. Org. Chem.; 37; 1972; 367).

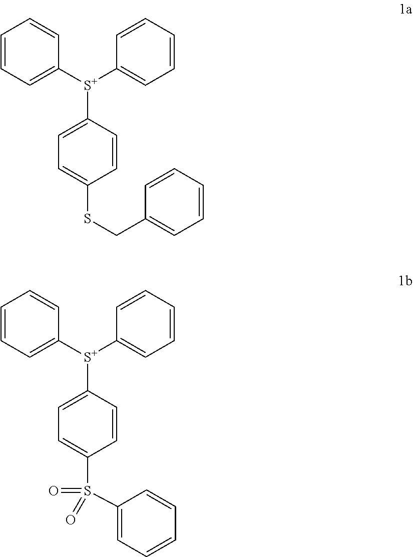

Also those sulphonium derivatives are described which contain three aromatic substituents linked to the S⁺ atom whereas two of the three substituents are bridged, see e.g. compound 2 (J. Org. Chem.; 70; 14; 2005; 5741-5744): (see scheme 2)

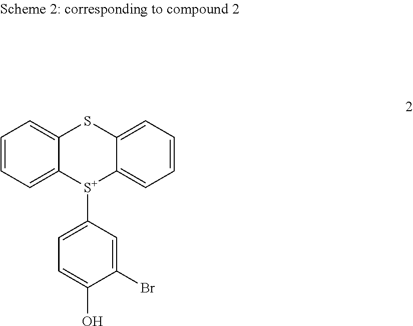

Some triaryl sulphonium derivatives are commercially available. For example para-halo triphenyl sulphonium salts are used as kationic photoinitiator (see scheme 3).

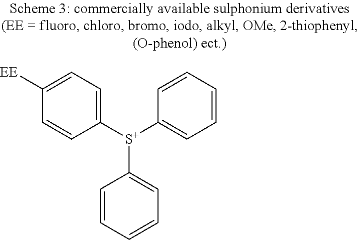

Also more complex sulphonium salts, like compound 3 (Apollo), are commercially offered.

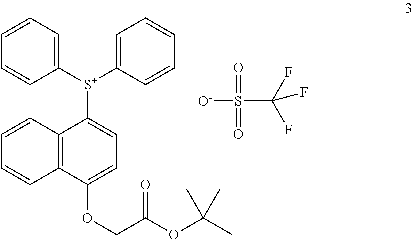

Ichikawa et al. (Chem. Lett. (1987), 1985) report on the F-19 fluorination of a methyl allyl sulfonium derivative which yields in a fluorinated (F-19) species in only “moderate yield” (Chem. Rev. (2008), 108, 1943-1981) probably due to generation of fluoro methan as side product.

It would be desirable to have methods available which allow the radioactive and non-radioactive fluorination of small and complex molecules comprising an aromatic moiety in good yield without forming side products containing the introduced fluoro atom in significant amounts.

This task is solved with the following invention:

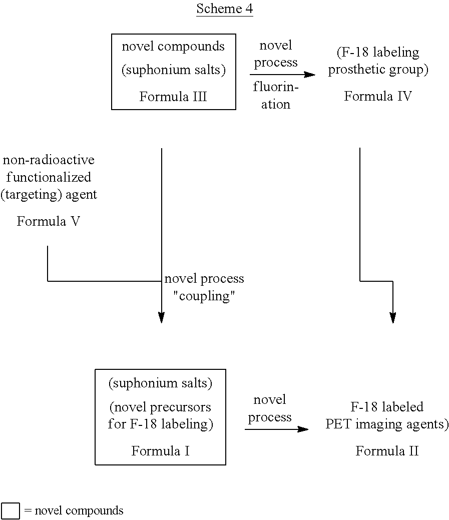

### SUMMARY

The present invention provides novel compounds of Formulae I and III.
The invention further provides a method of preparing compounds of formula II from compounds of formula I by reacting them with an appropriate F-fluorination agent.
The invention provides also a method of preparing compounds of formula IV characterized by reacting a compound of Formula III with an appropriate F-fluorination agent
The present invention provides also a method of preparing compounds of formula I by reacting compounds of formula III with compounds of general formula V
The invention also provides a kit for preparing a radiopharmaceutical preparation, said kit comprising a sealed vial containing a predetermined quantity of
   compound having formula I
   compounds of Formula III
   compounds of formulae III and V
The invention also provides a composition comprising a pharmaceutically acceptable carrier or diluent and
   a compound of formula I
   a compound of formula III
Another aspect of the present invention refers to the manufacturing of a medicament by using
   a compound of Formula I
   a compound of formula III

### DETAILED DESCRIPTION

The objects of the present invention are solved by a compound of formula I

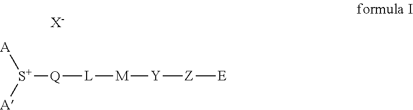

wherein A, A′ and Q are independently at each occurrence and individually selected from the group comprising aryl, substituted aryl, heteroaryl and substituted heteroaryl, wherein optionally either A and A′, A and Q or A′ and Q are linked to each other via R⁴,
   wherein, when substituted, there is one substituent or several substituents on said aryl or said heteroaryl, and the one or several substituent(s) is (are) located at any position of said aryl or heteroaryl,
   wherein S is sulfur,
   wherein X⁻ is selected from the group comprising a corresponding base of an inorganic acid and a corresponding base of an organic acid,
   wherein L-M-Y—Z is selected from the group comprising a bond and a linker, said bond being selected from a single, double or triple bond, said bond connecting E to Q, said linker connecting E to Q,
   wherein E is a targeting agent, and
   R⁴ being selected from the group comprising: bond, oxygen atom, sulphur atom, (N-alkyl) nitrogen, in particular (N—(C₁-C₄)alkyl)nitrogen, (C₁-C₃)alkylene, and (C₂-C₃)alkylene including all isomeric forms of said compound, including but not limited to enantiomers and diastereoisomers as well as racemic mixtures;
   and any pharmaceutically acceptable salt, ester, amide, complex or prodrug thereof.

In one embodiment, the invention is directed to compound of Formula I wherein the targeting agent is bound directly to a specific site in a biological system, such as an organism or a tissue, or in an in-vitro-system, such as a cell culture.

In one embodiment, the invention is directed to compound of Formula I wherein A and A′ are independently at each occurrence and individually selected from the group comprising
a) phenyl,
b) naphthyl
c) (C₁-C₆)alkyl phenyl,
d) halo phenyl,
e) (trifluoromethyl)phenyl,
f) methoxy phenyl,
g) hydroxyl phenyl,
h) cyano phenyl,
i) nitro phenyl,
j) ((C₁-C₆)alkyl sulphonyl)phenyl,
k) thienyl,
l) benzo[b]thienyl,
m) naphtho[2,3-b]thienyl,
n) thianthrenyl,
o) furyl,
p) pyranyl,
q) isobenzofuranyl,
r) benzoxazolyl,
s) chromenyl,
t) xanthenyl,
u) phenoxythiinyl,
v) 2H-pyrrolyl,
w) pyrrolyl,
x) imidazolyl,
y) pyrazolyl,
z) pyridyl,
aa) pyrazinyl,
bb) pyrimidinyl,
cc) pyridazinyl,
dd) indolizinyl,
ee) isoindolyl,
ff) 3H-indolyl,
gg) indolyl,
hh) indazolyl,
ii) purinyl,
jj) 4H-quinolizinyl,
kk) isoquinolyl,
ll) quinolyl,
mm) phthalazinyl,
nn) naphthyridinyl,
oo) quinazolinyl,
pp) cinnolinyl,
qq) pteridinyl,
rr) 4aH-carbazolyl,
ss) carbazolyl,
tt) carbolinyl,
uu) phenanthridinyl,
vv) acridinyl,
ww) perimidinyl,
xx) phenanthrolinyl,
yy) phenazinyl,
zz) isothiazolyl,
aaa) phenothiazinyl,
bbb) isoxazolyl,
ccc) furazanyl and
ddd) phenoxazinyl,
wherein either A and A′, A and Q or A′ and Q are linked to each other via R⁴.

Preferably, A and A′ are independently at each occurrence and individually selected from the group comprising
a) phenyl,
b) naphthyl
c) (C₁-C₂)alkyl phenyl,
d) halo phenyl,
e) (trifluoromethyl)phenyl,
f) methoxy phenyl,
g) cyano phenyl,
h) nitro phenyl,
i) ((C₁-C₂)alkyl sulphonyl)phenyl,
j) thienyl,
k) benzo[b]thienyl,
l) naphthol[2,3-b]thienyl,
m) thianthrenyl,
n) furyl,
o) pyranyl,
p) isobenzofuranyl,
q) benzoxazolyl,
r) chromenyl,
s) xanthenyl,
t) pyrrolyl,
u) imidazolyl,
v) pyrazolyl,
w) pyridyl,
x) pyrazinyl,
y) pyrimidinyl,
z) pyridazinyl,
aa) indolyl,
bb) indazolyl,
cc) isoquinolyl,
dd) quinolyl, and
ee) isothiazolyl,
   whereas either A and A′, A and Q or A′ and Q are linked to each other via R⁴.

More preferably, A and A′ are independently at each occurrence and individually selected from the group comprising
a) phenyl,
b) naphthyl
c) (C₁-C₂)alkyl phenyl,
d) fluoro phenyl
e) methoxy phenyl,
f) thienyl,
g) furyl,
h) pyranyl,
i) isobenzofuranyl, and
j) pyridyl.

Even more preferably, A and A′ are independently at each occurrence and individually selected from the group comprising
a) phenyl,
b) methyl phenyl,
c) methoxy phenyl,
d) thienyl, and
e) pyridyl.

Even more preferably, A and A′ are independently at each occurrence and individually selected from the group comprising
a) phenyl and
b) pyridyl.

In one embodiment, the invention is directed to compound of Formula I wherein Q is selected from the group comprising
a) phenyl,
b) (C₁-C₆)alkyl phenyl,
c) halo phenyl,
d) (trifluoromethyl)phenyl,
e) methoxy phenyl,
f) hydroxyl phenyl,
g) cyano phenyl,
h) nitro phenyl,
i) ((C₁-C₆)alkyl sulphonyl)phenyl
j) pyridyl and
k) naphthyl.

Preferably, Q is selected from the group comprising
a) phenyl,
b) (C₁-₃)alkyl phenyl,
c) fluoro phenyl,
d) (trifluoromethyl)phenyl,
e) cyano phenyl,
f) nitro phenyl and
g) ((C₁-C₃)alkyl sulphonyl)phenyl.

More preferably, Q is selected from the group comprising
a) phenyl,
b) (trifluoromethyl)phenyl,
c) cyano phenyl,
d) nitro phenyl and
e) (methyl sulphonyl)phenyl.

Preferably, Q is pyridyl.

Preferably, Q, when being substituted aryl or substituted heteroaryl, bears one or several substituents having positive 6 values as Hammett constant.

In one embodiment, the invention is directed to compound of Formula I wherein R⁴ is selected from the group comprising
a) bond,
b) oxygen atom,
c) sulphur atom,
d) (N—(C₁-C₄)alkyl)nitrogen,
e) (C₁-C₃)alkylene and
f) (C₂-C₃)alkenylen;

Preferably, R⁴ is selected from the group comprising
a) bond,
b) oxygen atom and
c) (C₁-C₃)alkylene;

More preferably, R⁴ is selected from the group comprising
a) bond,
b) oxygen atom, and
c) methylene.

In one embodiment, the invention is directed to compound of Formula I wherein X⁻ is selected from the group comprising
a) CH₃CH₂—O⁻,
b) CH₃—O⁻;
c) CF₃S(═O)₂O⁻,
d) H₃C—COO⁻,
e) C₄F₉S(═O)₂O⁻,
f) iodide anion,
g) bromide anion,
h) chloride anion,
i) perchlorate anion (ClO₄⁻),
j) phosphate anion, and
k) CF₃—COO⁻.

Preferably, X⁻ is selected from the group comprising
a) CF₃S(═O)₂O⁻,
b) H₃C—COO⁻,
c) C₄F₉S(═O)₂O⁻,
d) perchlorate anion (ClO₄⁻), and
e) CF₃—COO⁻.

More preferably, X⁻ is selected from the group comprising
a) CF₃S(═O)₂O⁻,
b) H₃C—COO⁻, and
c) CF₃—COO⁻

In one embodiment X⁻ is selected from the group comprising
a) CH₃CH₂—O⁻ and
b) CH₃—O⁻;

In another embodiment X⁻ is selected from the group comprising
a) CF₃S(═O)₂O⁻ and
b) H₃C—COO⁻,
c) C₄F₉S(═O)₂O⁻,
d) iodide anion,
e) bromide anion,
f) chloride anion,
g) perchlorate anion (ClO₄⁻),
h) phosphate anion, and
i) CF₃—COO⁻.

In one embodiment, the invention is directed to compound of Formula I wherein E is a targeting agent selected from the group comprising
a) peptide,
b) oligonucleotide and
c) small molecule.

Preferably, E is a targeting agent selected from the group comprising
a) peptide and
b) small molecule.

In one embodiment, the invention is directed to compound of Formula I wherein L-M-Y—Z is a linker, L is selected from the group comprising
a) —C(═O)H,
b) —S(═O)₂H,
c) —S(═O)H,
d) —N(H)—C(═O)H and
e) —C≡C—C(═O)—,
   wherein L is in ortho, meta, para or any other position to S⁺; and
   wherein M is selected from the group comprising
a) bond, said bond being selected from a single, double or triple bond,
b) —(CH₂)_{d}—,
c) —(CH₂)_{d}-D-(CH₂)_{d}—,
d) —N(R¹)—(CH₂)_{d}— and
e) —N(R¹)—(CH₂)ₚ—(CH₂—O—CH₂)ₖ—(CH₂)ₚ—;
   wherein D is selected from the group comprising aryl, substituted aryl, heteroaryl, substituted heteroaryl, and —N(R²)—; said R² being selected from the group comprising (C₁-C₆)alkyl, (C₁-C₆)alkyl aryl, aryl, substituted aryl, phenyl, substituted phenyl, heteroaryl, substituted heteroaryl and aralkyl;
   wherein the respective substituent on D is in ortho, meta or para-position, d is an integer from 0 to 6; p is an integer from 1 to 2 and k is an integer from 1 to 6; and
   wherein Y—Z is selected from the group comprising
a) C(═O)—O,
b) C(═O)—N(R¹),
c) O—C(═O),
d) N(R¹)—C(═O),
e) S(O)₂—N(R¹),
f) N(R¹)—S(═O)₂,
g) O—,
h) (S)ₚ—, p being an integer from 1 to 2,
i) N(R¹)—,
j) N(R¹)—C(═O)N(R¹),
k) N(R¹)C(═O)—N(R¹),
l) N(R¹)—C(═S)N(R¹),
m) N(R¹)C(═S)—N(R¹),
n) OC(═O)—O,
o) O—C(═O)O,
p) O—C(═S)O,
q) OC(═S)—O,
r) bond, said bond being selected from a single, double or triple bond, with the proviso that M and Y—Z are not both a bond at the same time, and

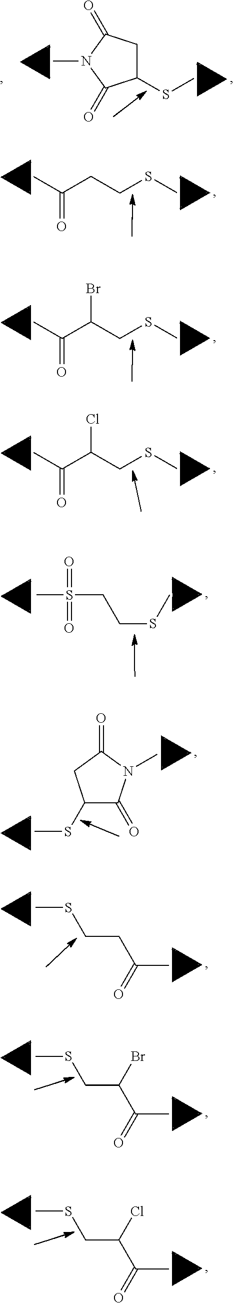

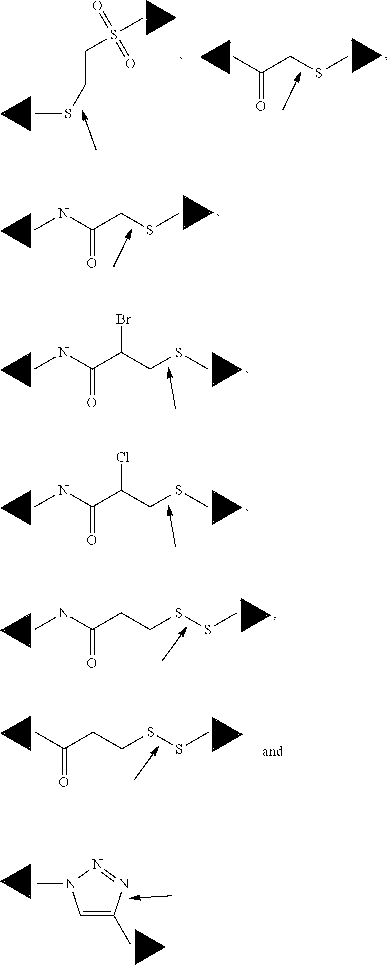

wherein the arrow indicates the bond between Z and Y and wherein Z is a reacted functional group of the targeting agent E, and wherein [symbol] . . . [symbol] denote M and the remainder of E devoid of said reacted functional group, respectively, and
wherein R¹, as defined for M and Y—Z, is independently at each occurrence selected from the group comprising hydrogen and (C₁-C₆)alkyl.

Preferably, L is selected from the group comprising
a) —C(═O)H,
b) —S(═O)₂H and
c) —N(H)—C(═O)H.

More preferably, L is selected from the group comprising
a) —C(═O)H and
b) —S(═O)₂H.

Preferably, M is selected from the group comprising
a) bond, said bond being selected from a single, double or triple bond,
b) —(CH₂)_{d}-D-(CH₂)_{d}—,
c) —N(R¹)—(CH₂)_{d}— and
d) —N(R¹)—(CH₂)ₚ—(CH₂—O—CH₂)ₖ—(CH₂)ₚ—;
   Wherein d, p, k, D, R¹ and R² being as defined above.

In one embodiment, the invention is directed to compound of Formula I wherein R¹ is selected from the group comprising
a) hydrogen and
b) (C₁-C₄)alkyl.

Preferably, R¹ is selected from the group comprising
a) hydrogen and
b) methyl.

In one embodiment, the invention is directed to compound of Formula I wherein R² is selected from the group comprising
a) phenyl,
b) (C₁-C₆)alkyl phenyl,
c) halo phenyl,
d) (trifluoromethyl)phenyl,
e) methoxy phenyl,
f) hydroxyl phenyl,
g) cyano phenyl,
h) nitro phenyl,
i) ((C₁-C₆)alkyl sulphonyl)phenyl,
j) pyridyl and
k) thienyl.

In one embodiment, the invention is directed to compound of Formula I wherein D is selected from the group comprising
a) phenyl,
b) (C₁-C₆)alkyl phenyl,
c) halo phenyl,
d) (trifluoromethyl)phenyl,
e) methoxy phenyl,
f) hydroxyl phenyl,
g) cyano phenyl,
h) nitro phenyl,
i) ((C₁-C₆)alkyl sulphonyl)phenyl,
j) pyridyl,
k) thienyl and
l) —N(R²)—.

Preferably, D is selected from the group comprising
a) phenyl,
b) —N(R²)—.

In one embodiment, the invention is directed to compound of Formula I wherein Y—Z is selected from the group comprising
a) C(═O)—O,
b) C(═O)—N(R¹),
c) O—C(═O),
d) N(R¹)—C(═O),
e) S(O)₂—N(R¹),
f) N(R¹)—,
g) N(R¹)—C(═O)N(R¹),
h) N(R¹)C(═O)—N(R¹),
i) N(R¹)—C(═S)N(R¹),
j) N(R¹)C(═S)—N(R¹), and

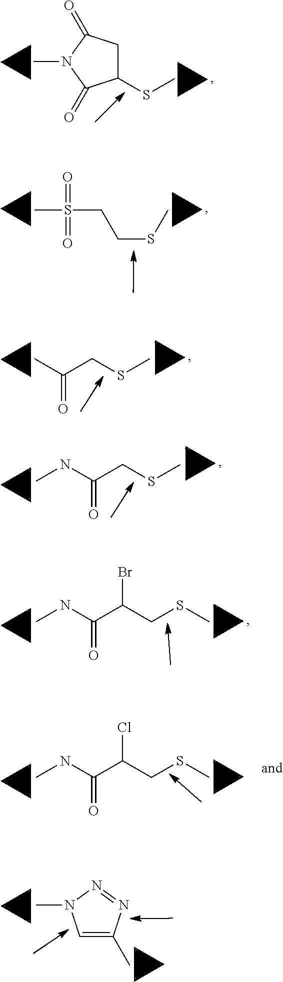

wherein the arrow indicates the bond between Z and Y and wherein Z is a reacted functional group of the targeting agent E, and wherein [symbol] . . . [symbol] and R¹ are as defined above.

Preferably, Y—Z is selected from the group comprising
a) C(═O)—O,
b) C(═O)—N(R¹),
c) O—C(═O),
d) N(R¹)—C(═O),
e) S(O)₂—N(R¹),
f) N(R¹)—S(═O)₂,
g) N(R¹)—,
h) N(R¹)—C(═O)N(R¹),
i) N(R¹)C(═O)—N(R¹),
j) N(R¹)—C(═S)N(R¹),
k) N(R¹)C(═S)—N(R¹), and

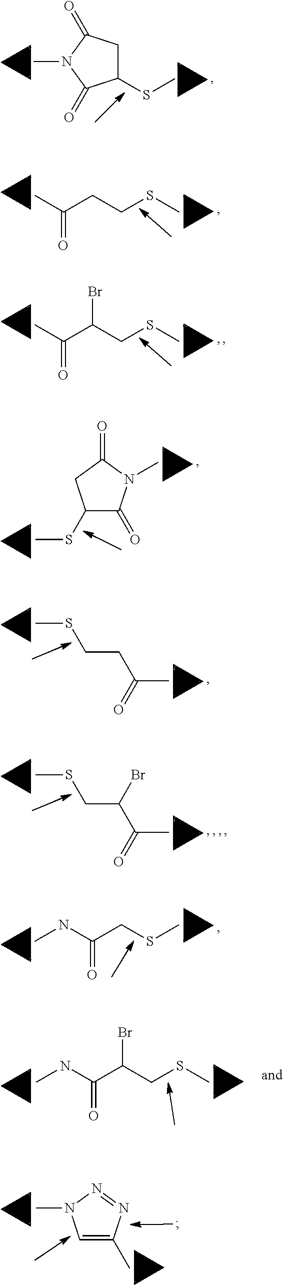

wherein the arrow indicates the bond between Z and Y and wherein Z is a reacted functional group of the targeting agent E, and wherein [symbol] . . . [symbol] and R¹ are as defined in any of claims 17-25.

Preferably, Y—Z is selected from the group comprising
a) C(═O)—O,
b) C(═O)—N(R¹),
c) N(R¹)—C(═O),
d) N(R¹)C(═O)—N(R¹),
e) N(R¹)C(═S)—N(R¹), and

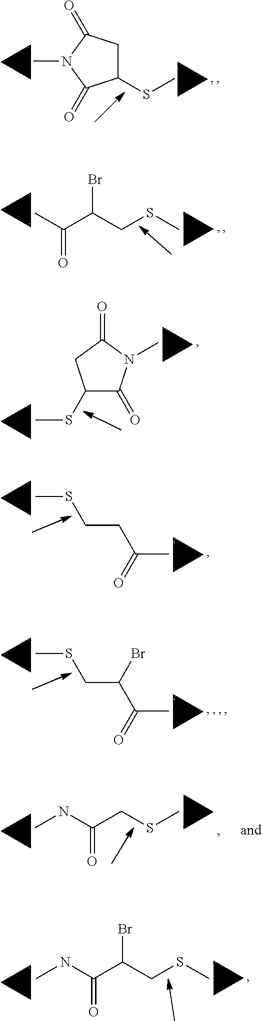

wherein the arrow indicates the bond between Z and Y and wherein Z is a reacted functional group of the targeting agent E, and wherein [symbol] . . . [symbol] and R¹ are as defined above.

The compound according to Formula I is selected from

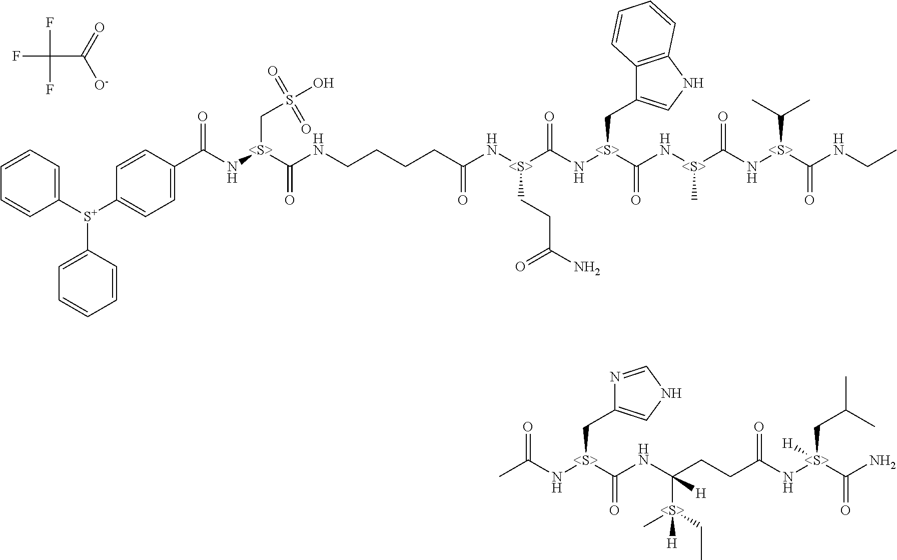

### 5-[(2R)-[4-Diphenylsulfoniumbenzoyl]-amino-1-oxo-sulfopropylamino]-1-oxopentyl-L-glutaminyl-L-tryptophanyl-L-alanyl-L-valyl-glycyl-L-histidinyl-[(4R)-amino-(5S)-methyl-heptanoyl]-L-leucine amide, trifluoroacetate

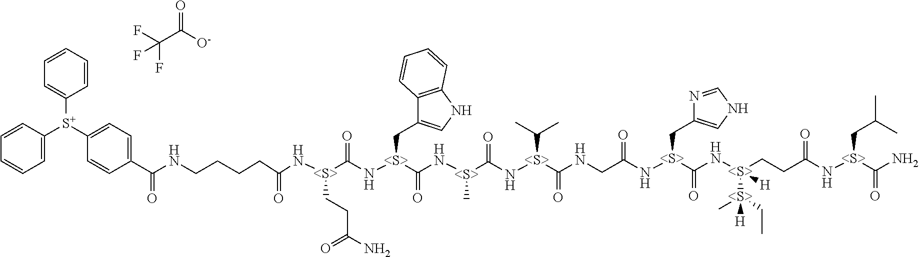

### 5-[4-Diphenylsulfoniumbenzoyl]-amino-1-oxopentyl-L-glutaminyl-L-tryptophanyl-L-alanyl-L-valyl-glycyl-L-histidinyl-[4-R-amino-5-S-methyl-heptanoyl]-L-leucine amide, trifluoroacetate

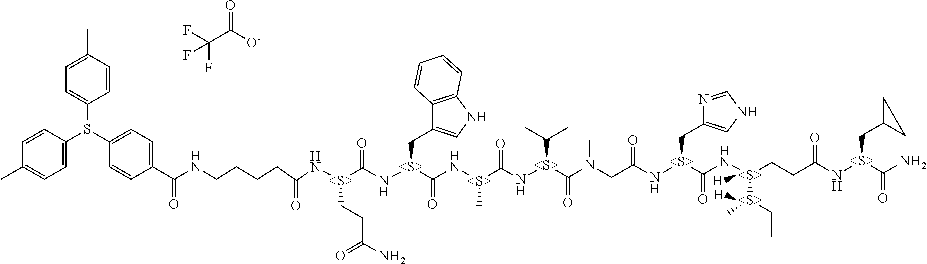

### 5-[4-Di-[4-methylphenyl]-sulfoniumbenzoyl]-amino-1-oxopentyl-L-glutaminyl-L-tryptophanyl-L-alanyl-L-valyl-N-methylglycyl-L-histidinyl-[4-R-amino-5-S-methyl-heptanoyl]-3-cyclopropylalanine amide, trifluoroacetate

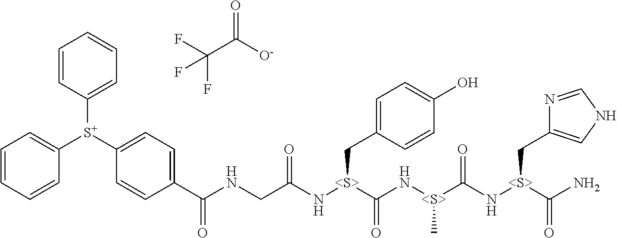

### 4-Diphenylsulfoniumbenzoyl-glycyl-L-tyrosinyl-L-alanyl-histidine amide, trifluoroacetate

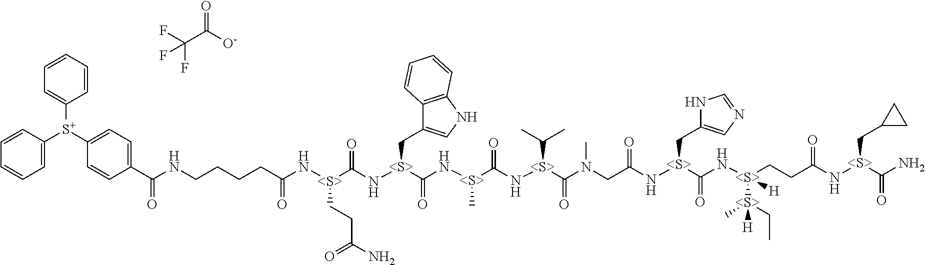

### 5-[4-Diphenylsulfoniumbenzoylamino]-1-oxopentyl-L-glutaminyl-L-tryptophanyl-L-alanyl-L-valyl-N-methylglycyl-L-histidinyl-[4-R-amino-5-S-methyl-heptanoyl]-3-cyclopropylalanine amide, trifluoroacetate

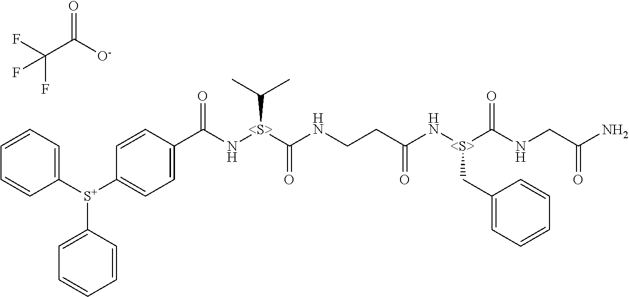

### 4-Diphenylsulfoniumbenzoyl-valyl-3-aminopropionyl-L-phenylalanyl-glycine amide, trifluoroacetate

These peptidic sulfonium salts shown above can be reacted with a fluorination agent, preferably a [¹⁸F]fluorination agent. Thus, sulfonium leaving groups are potent moieties to be labelled towards [¹⁸F] labelled biological molecules.

The objects of the present invention are also solved by a method of preparing a compound according to formula II

F-Q²-L-M-Y—Z-E
wherein L, M, Y, Z and E in formula II are as defined above, Q² being Q as defined above, and Q² being optionally substituted with —R⁴(A) wherein R⁴ and A are as defined above, and wherein F is selected from the group comprising [¹⁸F]fluoro and [¹⁹F]fluoro, and
wherein a compound of formula I, as defined above, is reacted with a fluorination agent, said fluorination agent being a chemical agent comprising fluoride anions, wherein said fluoride anion is of an [¹⁸F]fluoro or [¹⁹F]fluoro isotope.

In other words, the method for obtaining compounds of Formula II as defined above comprises the step
Reacting compound of Formula I with fluorination agent.

The objects of the present invention are also solved by a compound according to formula III

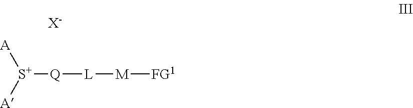

wherein A, A′, S, Q, L, X⁻ and M are as defined above, and
wherein FG¹ is selected from the group comprising
a) hydroxy,
b) iodo,
c) bromo,
d) chloro,
e) N₃,
f) C≡CH,
g) C(O)OR³,
h) active ester moiety,
i) C(O)-Hal,
j) NHR¹,
k) N═C═O,
l) N═C═S,
m) O—-S(O)₂-aryl,
n) OS(O)₂-alkyl,
o) SO₂-Hal,
p) S3H,
q) SH,
r) O—C(═O)-Hal,
s) O—C(═S)-Hal,
t)

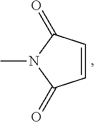

u)

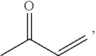

v)

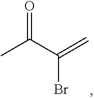

w)

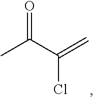

x)

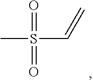

y)

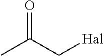

and
z)

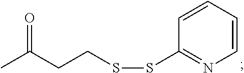

wherein R¹ is as defined above, and wherein R³ is selected from the group comprising
a) hydrogen,
b) active ester moiety,
c) (C₁-C₆)alkyl,
d) (C₂-C₆)alkenyl and
e) aryl alkyl; and
   wherein Hal is a halogen, such as F, Cl, Br or I
      including all isomeric forms of said compound, including but not limited to enantiomers and diastereoisomers as well as racemic mixtures;
      and any pharmaceutically acceptable salt, ester, amide, complex or prodrug thereof.
In one embodiment, the invention is directed to compound of Formula III wherein FG¹ is selected from the group comprising
   a) hydroxy,
   b) bromo,
   c) N₃,
   d) C≡CH,
   e) C(O)OH
   f) NHR¹,
   g) N═C═O,
   h) N═C═S,
   i) S(O)₂—Cl,
   j) SH,
   k) O—C(═O)-Hal,
   l) O—C(═S)-Hal,
   m)

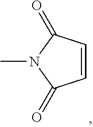

n)

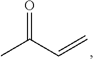

o)

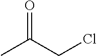

and
p)

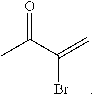

Preferably, FG¹ is selected from the group comprising
a) hydroxy,
b) N₃,
c) C≡CH,
d) C(O)OH
e) NHR¹,
f) N═C═S,
g) SH,
h)

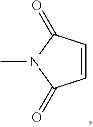

i)

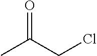

and
j)

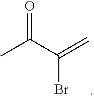

In one embodiment, the invention is directed to compound of Formula III wherein R³ is selected from the group comprising
   a) hydrogen,
   b) (C₁-C₆)alkyl,
   c) (C₂-C₆)alkenyl,
   d) aryl alkyl and
   e)

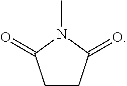

Preferably, R³ is selected from the group comprising
a) hydrogen,
b) (C₁-C₃)alkyl,
c) (C₂-C₃)alkenyl,
d) phenyl(C1-C6)alkyl and

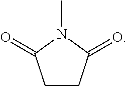

More preferably, R³ is selected from the group comprising
a) hydrogen,
b) methyl,
c) benzyl and

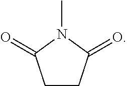

The compound according to Formula III is selected from the group comprising

| | |
|---|---|
| (4-carboxyphenyl)[bis(4- methylphenyl)[sulfonium trifluoromethanesulfonate | 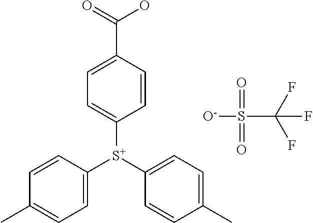 |
| 4- carboxyphenyl)(diphenyl)sulfonium trifluoromethanesulfonate | 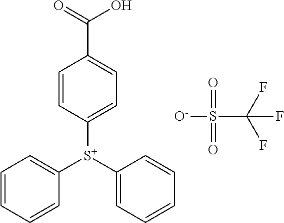 |
| [4-carboxy-3- (trifluoromethyl)phenyl](diphenyl)sulfonium trifluoromethanesulfonate | 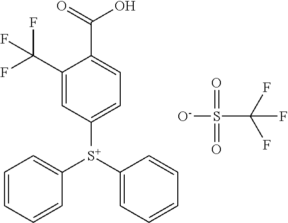 |
| (5-carboxypyridin-2- yl)(diphenyl)sulfonium trifluoromethanesulfonate | 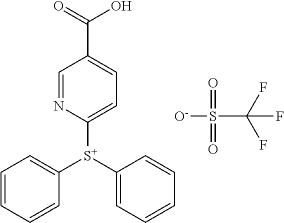 |
| (6-carboxypyridin-2- yl)(diphenyesulfonium trifluoromethanesulfonate | 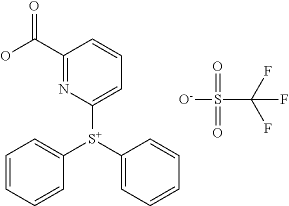 |
| (2-carboxypyridin-4- yl)(diphenyl)sulfonium trifluoromethanesulfonate | 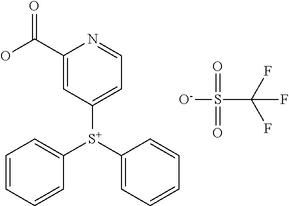 |
| [4- (methoxycarbonyl)phenyl](diphenyl)sulfonium trifluoromethanesulfonate | 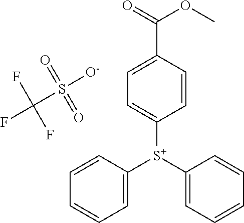 |

The objects of the present invention are also solved by a method of preparing a compound of formula IV

¹⁸F-Q²-L-M-FG¹ IV,

wherein

### Q² is Q;

L is selected from the group comprising
a) —C(═O)H,
b) —S(═O)₂H,
c) —S(═O)H,
d) —N(H)—C(═O)H and
e) —C≡C(═O)—,
   wherein L is in ortho, meta, para or any other position to S⁺; and
   wherein M is selected from the group comprising
a) bond, said bond being selected from a single, double or triple bond,
b) —(CH₂)_{d}—,
c) —(CH₂)_{d}-D-(CH₂)_{d}—,
d) —N(R¹)—(CH₂)_{d}— and
e) —N(R¹)—(CH₂)ₚ—(CH₂—O—CH₂)ₖ—(CH₂)ₚ—;
FG¹ is selected from the group comprising
a) hydroxy,
b) iodo,
c) bromo,
d) chloro,
e) N₃,
f) C≡CH,
g) C(O)OR³,
h) active ester moiety,
i) C(O)-Hal,
j) NHR¹,
k) N═C═O,
l) N═C═S,
m) O—S(O)₂-aryl,
n) OS(O)₂-alkyl,
o) SO₂-Hal,
p) S3H,
q) SH,
r) O—C(═O)-Hal,
s) O—C(═S)-Hal,
t)

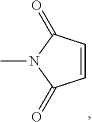

u)

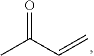

v)

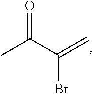

w)

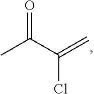

x)

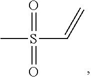

y)

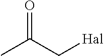

and
z)

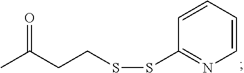

wherein ¹⁸F is the [¹⁸F]-fluorine isotope, wherein a compound of formula III, as defined above is reacted with a fluorination agent, said fluorination agent being a chemical agent comprising fluoride anions, wherein said fluoride anion is of an [¹⁸F] or [¹⁹F] isotope.

In other words, the method for obtaining compounds of Formula IV as defined above comprises the step
Reacting compound of Formula III with a fluorination agent.

The objects of the present invention are also solved by a method of preparing a compound according to formula I, as defined above, wherein a compound according to formula III as defined above is reacted with a compound according to formula V

E-FG² V,

wherein FG² is identical to FG¹ as defined above, and wherein E is as defined above, and wherein FG¹ and FG² are independently at each occurrence selected from the group as defined above and are selected such that, upon reaction, they establish Y—Z as defined above.

In other words, the method for obtaining compounds of Formula I as defined above comprises the step
Reacting compound of Formula III with a compound according to formula V.

The objects of the present invention are also solved by a composition comprising a compound according to formula I as defined above, or a compound according to formula III as defined above, and a pharmaceutically acceptable carrier or diluent.

The objects of the present invention are also solved by a kit comprising a sealed vial containing a predetermined quantity of a compound according to formula I, as defined above, or of a compound according to formula III as defined above.

Preferably, the kit according to the present invention comprises a sealed vial containing a predetermined quantity of a compound according to formula I, as defined above, and a sealed vial containing a predetermined quantity of a fluorination agent, said fluorination agent being a chemical agent comprising fluoride anions, wherein said fluoride anion is of an [¹⁸F] isotope.

In one embodiment, the kit according to the present invention further comprises a sealed vial containing a predetermined quantity of a compound of a fluorination agent, said fluorination agent being a chemical agent comprising fluoride anions, wherein said fluoride anion is of an [¹⁹F] isotope.

In one embodiment, the kit according to the present invention comprises a sealed vial containing a predetermined quantity of a compound according to formula III as defined above, and a sealed vial containing a predetermined quantity of a compound according to formula V

E-FG²

as defined above.

In one embodiment, the kit according to the present invention further comprises a sealed vial containing a predetermined quantity of a fluorination agent, said fluorination agent being a chemical agent comprising fluoride anions, wherein said fluoride anion is of an [¹⁸F] isotope.

In one embodiment, the kit according to the present invention further comprises a sealed vial containing a predetermined quantity of a fluorination agent, said fluorination agent being a chemical agent comprising fluoride anions, wherein said fluoride anion is of an [¹⁹F] isotope.

The objects of the present invention are also solved by the use of a compound according to formula I, II, III and IV as defined above or of a composition as defined above, for the manufacture of a medicament for the treatment of CNS diseases including but not limited to inflammatory and autoimmune, allergic, infectious and toxin-triggered and ischemia-triggered diseases, pharmacologically triggered inflammation with pathophysiological relevance, neuroinflammatory, neurodegenerative diseases, cancers, cardiovascular diseases, and metabolic diseases.

The objects of the present invention are also solved by the use of compounds of Formula II or IV as imaging agent.

Preferably, the imaging agent is useful for PET, SPECT or Micro-PET imaging. More

Preferably, the imaging agent is useful for PET imaging.

Preferably, the imaging agent is suitable for imaging CNS diseases including but not limited to inflammatory and autoimmune, allergic, infectious and toxin-triggered and ischemia-triggered diseases, pharmacologically triggered inflammation with pathophysiological relevance, neuroinflammatory, neurodegenerative diseases, cancers, cardiovascular diseases, and metabolic diseases.

The present invention is also directed to a method for imaging diseases, as defined above, comprising the step of introducing into a patient a detectable quantity of radiolabelled compound having Formula II or IV. Additionally, radiations are measured or signal is detected and diagnostic can be established. In other words, the signal is detected.

The objects of the present invention are also solved by a method for staging, monitoring of hyperproliferative disease progression, or monitoring response to therapy directed to hyperproliferative diseases using compounds of the invention.

It should be clear to someone skilled in the art that the compounds in accordance with formula I may be labeled by any suitable positron emitting isotope, such as ¹¹C, ¹³N, ¹⁵O and ¹⁸F, with, however, ¹⁸F being preferred due to the longer half life. Hence, whilst in the major part of this application, labeling with ¹⁸F is described, it should be understood that this is only a preferred embodiment. The compounds in accordance with formula III and formula I may contain positron emitting isotopes other than ¹⁸F. In such a case, they may additionally get labeled with an ¹⁸F fluorinating agent or they may get fluorinated by the corresponding cold ¹⁹F fluorinating agent. Hence, formulae I, II, III, IV and V, as depicted above, do not exclude the presence of positron emitting isotopes other than ¹⁸F.

For the purposes of the present invention, the term “targeting agent” shall have the following meaning: The targeting agent is a compound or moiety that targets or directs the radionuclide attached to it to a specific site in a biological system. A targeting agent can be any compound or chemical entity that binds to or accumulates at a target site in a mammalian body, i.e., the compound localizes to a greater extent at the target site than to surrounding tissue.

For the purpose of the present invention, the term “peptide” refers to a molecule comprising an amino acid sequence of at least two amino acids.

For the purpose of the present invention the term “amino acid sequence” is defined herein as a polyamide obtainable by polycondensation of at least two amino acids. For the purpose of the present invention the term “amino acid” means any molecule comprising at least one amino group and at least one carboxyl group, but no peptide bond within the molecule. In other words, an amino acid is a molecule that has a carboxylic acid functionality and an amine nitrogen having at least one free hydrogen, preferably in alpha position thereto, but no amide bond in the molecule structure. Thus, a dipeptide having a free amino group at the N-terminus and a free carboxyl group at the C-terminus is not to be considered as a single “amino acid” in the above definition.

An amide bond as used herein means any covalent bond having the structure

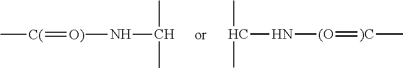

wherein the carbonyl group is provided by one molecule and the NH-group is provided by the other molecule to be joined. The amide bonds between two adjacent amino acid residues which are obtained from such a polycondensation are defined as “peptide bonds”. Optionally, the nitrogen atoms of the polyamide backbone (indicated as NH above) may be independently alkylated, e.g. with —C₁-C₆-alkyl, preferably —CH₃.

For the purpose of the specification an amino acid residue is derived from the corresponding amino acid by forming a peptide bond with another amino acid.

For the purpose of the specification an amino acid sequence may comprise naturally occurring and/or artificial amino acid residues, proteinogenic and/or non-proteinogenic amino acid residues. The non-proteinogenic amino acid residues may be further classified as (a) homo analogues of proteinogenic amino acids, (b) β-homo analogues of proteinogenic amino acid residues and (c) further non-proteinogenic amino acid residues.

Accordingly, the amino acid residues are derived from the corresponding amino acids, e.g. from
proteinogenic amino acids, namely Ala, Arg, Asn, Asp, Cys, Gln, Glu, Gly, His, Ile, Leu, Lys, Met, Phe, Pro, Ser, Thr, Trp, Tyr and Val; or
non-proteinogenic amino acids, such as
   homo analogues of proteinogenic amino acids wherein the sidechain has been extended by a methylene group, e.g. Homoalanine (Hal), Homoarginine (Har), Homocysteine (Hcy), Homoglutamine (Hgl), Homohistidine (Hhi), Homoisoleucine (Hil), Homoleucine (Hle), Homolysin (Hly), Homomethionine (Hme), Homophenylalanine (Hph), Homoproline (Hpr), Homoserine (Hse), Homothreonine (Hth), Homotryptophane (Htr), Homotyrosine (Hty) and Homovaline (Hva);
   β-homo analogues of proteinogenic amino acids wherein a methylene group has been inserted between the α-carbon and the carboxyl group yielding β-amino acids, e.g. β-Homoalanine (βHal), β-Homoarginine (βHar), β-Homoasparagine (βHas), β-Homocysteine (βHcy), β-Homoglutamine (βHgl), β-Homohistidine (βHhi), β-Homoisoleucine (βHil), β-Homoleucine (βHle), β-Homolysine (βHly), β-Homomethionine (βHme), β-Homophenylalanine (βHph), β-Homoproline (βHpr), β-Homoserine (βHse), β-Homothreonine (βHth), β-Homotryptophane (βHtr), β-Homotyrosine (βHty) and β-Homovaline (βHva);
   further non-proteinogenic amino acids, e.g. α-Aminoadipic acid (Aad), β-Aminoadipic acid (βAad), α-Aminobutyric acid (Abu), α-Aminoisobutyric acid (Aib), β-Alanine (βAla), 4-Aminobutyric acid (4-Abu), 5-Aminovaleric acid (5-Ava), 6-Aminohexanoic acid (6-Ahx), 8-Aminooctanoic acid (8-Aoc), 9-Aminononanoic acid (9-Anc), 10-Aminodecanoic acid (10-Adc), 12-Aminododecanoic acid (12-Ado), α-Aminosuberic acid (Asu), Azetidine-2-carboxylic acid (Aze), β-Cyclohexylalanine (Cha), Citrulline (Cit), Dehydroalanine (Dha), γ-Carboxyglutamic acid (Gla), α-Cyclohexylglycine (Chg), Propargylglycine (Pra), Pyroglutamic acid (Glp), α-tert-Butylglycine (Tle), 4-Benzoylphenylalanine (Bpa), δ-Hydroxylysine (Hyl), 4-Hydroxyproline (Hyp), allo-Isoleucine (aIle), Lanthionine (Lan), (1-naphthyl)alanine (1-Nal), (2-naphthyl)alanine (2-Nal), Norleucine (Nle), Norvaline (Nva), Ornithine (Orn), Phenylglycin (Phg), Pipecolic acid (Pip), Sarcosine (Sar), Selenocysteine (Sec), Statine (Sta), β-Thienylalanine (Thi), 1,2,3,4-Tetrahydroisochinoline-3-carboxylic acid (Tic), allo-Threonine (aThr), Thiazolidine-4-carboxylic acid (Thz), β-Aminobutyric acid (GABA), iso-Cysteine (iso-Cys), Diaminopropionic acid (Dpr), 2,4-Diaminobutyric acid (Dab), 3,4-Diaminobutyric acid (γ,βDab), Biphenylalanine (Bip), Phenylalanine substituted in para-position with —C₁-C₆-alkyl, -halide, —NH₂ or —CO₂H (Phe(4-R) wherein R=C₁-C₆-alkyl, -halide, —NH₂, or —CO₂H); peptide nucleic acids (PNA, cf. P. E. Nielsen, Acc. Chem. Res. 32, 624-30)
or their N-alkylated analogues, such as their N-methylated analogues.

Cyclic amino acids may be proteinogenic or non-proteinogenic, such as Pro, Aze, Glp, Hyp, Pip, Tic and Thz.

For further examples and details reference can be made to e.g. J. H. Jones, J. Peptide Sci. 2003, 9, 1-8 which is incorporated herein by reference.

The terms “non-proteinogenic amino acid” and “non-proteinogenic amino acid residue” also encompasses derivatives of proteinogenic amino acids. For example, the sidechain of a proteinogenic amino acid residue may be derivatized thereby rendering the proteinogenic amino acid residue “non-proteinogenic”. The same applies to derivatives of the C-terminus and/or the N-terminus of a proteinogenic amino acid residue terminating the amino acid sequence.

For the purpose of the specification a proteinogenic amino acid residue is derived from a proteinogenic amino acid selected from the group consisting of Ala, Arg, Asn, Asp, Cys, Gln, Glu, Gly, His, Ile, Leu, Lys, Met, Phe, Pro, Ser, Thr, Trp, Tyr and Val either in L- or D-configuration; the second chiral center in Thr and Ile may have either R- or S-configuration. Therefore, for example, any posttranslational modification of an amino acid sequence, such as N-alkylation, which might naturally occur renders the corresponding modified amino acid residue “non-proteinogenic”, although in nature said amino acid residue is incorporated in a protein.

For the purposes of this invention, the term “small molecule” shall have the following meaning: a small molecule is a compound that has a molecular mass of 200 to 800 and that contains a functional group to which compounds of Formula III and V are coupled as Y—Z. Such targeting moieties are known in the art, so are methods for preparing them.

Preferably the targeting agent E is a peptide (or a peptidomimetic) or an oligonucleotide or a small molecule, particularly one which has specificity to target the complex to a specific site in a biological system. Smaller organic molecules effective for targeting certain sites in a biological system can also be used as the targeting agent.

Small molecules effective for targeting certain sites in a biological system can be used as the targeting agent E. Smaller organic molecules may be “small chemical entities”. As used in this application, the term “small chemical entity” shall have the following meaning: A small chemical entity is a compound that has a molecular mass of from 200 to 800 or of from 150 to 700, more preferably from 200 to 700, more preferably from 250 to 700, even more preferably from 300 to 700, even more preferably from 350 to 700 and most preferably from 400 to 700. A small chemical entity as used herein may further contain at least one aromatic or heteroaromatic ring and may also have a primary or secondary amine via which the benzene ring structure in the compounds of general chemical Formulae I and II is coupled via -L-Y—. Such targeting moieties are known in the art, so are methods for preparing them.

The small molecule targeting agents may preferably be selected from those described in the following references: P. L. Jager, M. A. Korte, M. N. Lub-de Hooge, A. van Waarde, K. P. Koopmans, P. J. Perik and E. G. E. de Vries, Cancer Imaging, (2005) 5, 27-32; W. D. Heiss and K. Herholz, J. Nucl. Med., (2006) 47(2), 302-312; and T. Higuchi and M. Schwaiger, Curr. Cardiol. Rep., (2006) 8(2), 131-138. More specifically examples of small molecule targeting agents are listed hereinafter:

| Name | Abbr. | target |
|---|---|---|
| ¹⁸F-2b-Carbomethoxy-3b-(4- | CFT | DAT (dopamine transporter) |
| fluorophenyl)tropane | | |
| ¹⁸F-Fluoroethylspiperone | FESP | D2 (dopamine 2 receptor), 5-HT₂ |
| | | (5-hydroxytryptamine receptor) |
| ¹⁸F-Fallypride | | D2 (dopamine 2 receptor) |
| ¹⁸F-Altanserin | | 5-HT2A receptor |
| ¹⁸F-Cyclofoxy | | Opioid receptors |
| ¹⁸F-CPFPX | | Adenosine A1 receptor |
| Batimastat | | MMP |
| Fatty acids and analogues | | |
| Choline analogues (metabolism) | | |
| Flumazenil | | Benzodiazepine receptors |
| Raclopride | | D2 receptors |
| Dihydrotestosteron and analogues | | AR |
| Tamoxifen and analogues | | |
| Deoxyglucose | | |
| Thymidine | | Proliferation - thymidine kinase |
| DOPA | | |
| Benzazepines | | D₁ antagonists |
| N-methyl spiperone and | | dopamine receptors |
| derivatives thereof | | |
| Benzamide raclopride; benzamide | | D₂ receptors |
| derivatives, e.g., fallopride, iodo | | |
| benzamide; clozapine, quietapine | | |
| Nomifensine, substituted analogs | | DAT |
| of cocaine, e.g., tropane type | | |
| derivatives of cocaine, methyl | | |
| phenidate | | |
| 2β-Carboxymethoxy-3β-(4- | CIT | DAT |
| iodophenyl)tropane | | |
| | CIT-FE, CIT-FM | DAT |
| Altanserin, setoperon, ketanserin | | 5-HT_{2A} |
| | McN5652, 403U76 derivative | 5-HTT |
| | ADAM, DASP, MADAM | |
| Acetylcholine analogues | MP3A, MP4A, PMP; QNB, TKB, | acetylcholine receptors |
| | NMPB, | |
| Scopolamine, benztropine | | acetylcholine receptors |
| Flumazenil | | GABA receptor |
| | RO-15-4513, FDG | GABA receptor |
| | PK-11195 | benzodiazepine receptor |
| Xanthine analogues | CPFPX, MPDX | adenosine receptor |
| Carfentanyl, diprenorphine | | opoid receptor |

In another preferred embodiment the targeting agent E is a peptide.

The targeting agent E may be a peptide comprising from 4 to 100 amino acids wherein the amino acids may be selected from natural and non-natural amino acids and also may comprise modified natural and non-natural amino acids.

Examples for peptides as targeting agent E are, but are not limited to, somatostatin and derivatives thereof and related peptides, somatostatin receptor specific peptides, neuropeptide Y and derivatives thereof and related peptides, neuropeptide Y₁ and the analogs thereof, bombesin and derivatives thereof and related peptides, gastrin, gastrin releasing peptide and the derivatives thereof and related peptides, epidermal growth factor (EGF of various origin), insulin growth factor (IGF) and IGF-1, integrins (α₃β₁, αᵥβ₃, αᵥβ₅, αIIb₃), LHRH agonists and antagonists, transforming growth factors, particularly TGF-α; angiotensin; cholecystokinin receptor peptides, cholecystokinin (CCK) and the analogs thereof; neurotensin and the analogs thereof, thyrotropin releasing hormone, pituitary adenylate cyclase activating peptide (PACAP) and the related peptides thereof, chemokines, substrates and inhibitors for cell surface matrix metalloproteinase, prolactin and the analogs thereof, tumor necrosis factor, interleukins (IL-1, IL-2, IL-4 or IL-6), interferons, vasoactive intestinal peptide (VIP) and the related peptides thereof. Such peptides comprise from 4 to 100 amino acids, wherein the amino acids are selected from natural and non-natural amino acids and also comprise modified natural and non-natural amino acids. Preferably targeting agent E is not insulin.

More preferably targeting agent E may be selected from the group comprising bombesin and bombesin analogs, preferably those having the sequences listed herein below, somatostatin and somatostatin analogs, preferably those having the sequences listed herein below, neuropeptide Y₁ and the analogs thereof, preferably those having the sequences listed herein below, vasoactive intestinal peptide (VIP) and the analogs thereof.

Even more preferably targeting agent E may be selected from the group comprising bombesin, somatostatin, neuropeptide Y₁ and the analogs thereof.

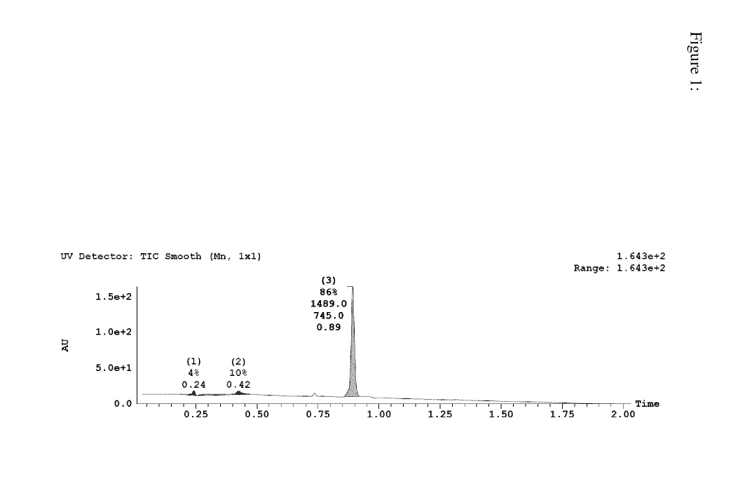

Further various small molecule targeting agents and the targets thereof are given in Table 1 in W. D. Heiss and K. Herholz, ibid. and in Figure 1 in T. Higuchi, M. Schwaiger, ibid.

In another preferred embodiment targeting agent (E) may be selected from the group comprising oligonucleotides comprising from 4 to 100 nucleotides.

In other preferred embodiments the targeting agent E is selected to be an oligonucleotide. In a further preferred embodiment the targeting agent E may be selected from the group comprising oligonucleotides comprising from 4 to 100 nucleotides.

In another preferred embodiment E is selected to be a peptide comprising from 4 to 100 amino acids or to be a oligonucleotide comprising from 4 to 100 nucleotides or peptidomimetics.

For the purposes of this invention, the term “oligonucleotide” shall have the following meaning: short sequences of nucleotides, typically with twenty or fewer bases. Examples are, but are not limited to, molecules named and cited in the book: “The aptamers handbook. Functional oligonuclides and their application” by Svenn Klussmann, Wiley-VCH, 2006. An example for such an oligonucleotide is TTA1 (J. Nucl. Med., 2006, April, 47(4), 668-78). For the purpose of this invention, the term “aptamer” refers to an oligonucleotide, comprising from 4 to 100 nucleotides, wherein at least two single nucleotides are connected to each other via a phosphodiester linkage. Said aptamers have the ability to bind specifically to a target molecule (see e.g. M Famulok, G Mayer, Aptamers as Tools in Molecular Biology and Immunology, In: Combinatorial Chemistry in Biology, Current Topics in Microbiology and Immunology (M Famulok, C H Wong, E L Winnacker, Eds.), Springer Verlag Heidelberg, 1999, Vol. 243, 123-136). There are many ways known to the skilled person of how to generate such aptamers that have specificity for a certain target molecule. An example is given in WO 01/09390, the disclosure of which is hereby incorporated by reference. Said aptamers may comprise substituted or non-substituted natural and non-natural nucleotides. Aptamers can be synthesized in vitro using e.g. an automated synthesizer. Aptamers according to the present invention can be stabilized against nuclease degradation e.g. by the substitution of the 2′-OH group versus a 2′-fluoro substituent of the ribose backbone of pyrimidine and versus 2′-O-methyl substituents in the purine nucleic acids. In addition, the 3′ end of an aptamer can be protected against exonuclease degradation by inverting the 3′ nucleotide to form a new 5′-OH group, with a 3′ to 3′ linkage to a penultimate base.

For the purpose of this invention, the term “nucleotide” refers to molecules comprising a nitrogen-containing base, a 5-carbon sugar, and one or more phosphate groups. Examples of said base comprise, but are not limited to, adenine, guanine, cytosine, uracil, and thymine Also non-natural, substituted or non-substituted bases are included. Examples of 5-carbon sugar comprise, but are not limited to, D-ribose, and D-2-desoxyribose. Also other natural and non-natural, substituted or non-substituted 5-carbon sugars are included. Nucleotides as used in this invention may comprise from one to three phosphates.

In one embodiment, the targeting agent E is a biologically active molecule which has a binding affinity to a biological target.

In one embodiment the targeting agent E is a biologically active molecule which has a binding affinity to a biological target being relevant in CNS or oncological or cardiovascular diseases.

In one embodiment the targeting agent E is a biologically active molecule which has a binding affinity to a biological target being relevant in CNS diseases.

In one embodiment the targeting agent E is a biologically active molecule which has a binding affinity to a biological target being relevant in an oncological diseases.

In one embodiment the targeting agent E is a biologically active molecule which has a binding affinity to a biological target being relevant in a cardiovascular diseases.

In one embodiment the targeting agent E is a biologically active molecule which has a binding affinity to a biological target which is smaller than 10 micro molar.

In a preferred embodiment the targeting agent E is a biologically active molecule which has a binding affinity to a biological target which is smaller than 1 micro molar.

In a preferred embodiment the targeting agent E is a biologically active molecule which has a binding affinity to a biological target which is smaller than 500 nM.

In a preferred embodiment the targeting agent E is a biologically active molecule which has a binding affinity to a biological target which is smaller than 100 nM.

In a preferred embodiment the targeting agent E is a biologically active molecule which has a binding affinity to a biological target which is smaller than 75 nM.

In a preferred embodiment the targeting agent E is a biologically active molecule which has a binding affinity to a biological target which is smaller than 50 nM.

In a preferred embodiment the targeting agent E is a biologically active molecule which has a binding affinity to a biological target which is smaller than 30 nM.

In a preferred embodiment the targeting agent E is a biologically active molecule which has a binding affinity to a biological target which is smaller than 15 nM.

In a preferred embodiment the targeting agent E is a biologically active molecule which has a binding affinity to a biological target which is smaller than 10 nM.

In a preferred embodiment the targeting agent E is a biologically active molecule which has a binding affinity to a biological target which is smaller than 5 nM.

In a preferred embodiment the targeting agent E is a biologically active molecule which has a binding affinity to a biological target which is smaller than 2 nM.

In a preferred embodiment the targeting agent E is a biologically active molecule which has a binding affinity to a biological target which is smaller than 1 nM.

In a preferred embodiment the targeting agent E is a biologically active molecule which has a binding affinity to a biological target which is smaller than 750 piko molar.

In a preferred embodiment the targeting agent E is a biologically active molecule which has a binding affinity to a biological target which is smaller than 500 piko molar.

In a preferred embodiment the targeting agent E is a biologically active molecule which has a binding affinity to a biological target which is smaller than 250 piko molar.

In a preferred embodiment the targeting agent E is a biologically active molecule which has a binding affinity to a biological target which is smaller than 100 piko molar.

In a preferred embodiment the targeting agent E is a biologically active molecule which has a binding affinity to a biological target which is smaller than 50 piko molar.

In one embodiment E is selected from the group of molecules which have a mass higher than 50;
In one embodiment E is selected from the group of molecules which have a mass higher than 70;
In one embodiment E is selected from the group of molecules which have a mass higher than 85;
In one embodiment E is selected from the group of molecules which have a mass higher than 100;
In one embodiment E is selected from the group of molecules which have a mass higher than 120;
In one embodiment E is selected from the group of molecules which have a mass higher than 140;
In one embodiment E is selected from the group of molecules which have a mass higher than 160;
In one embodiment E is selected from the group of molecules which have a mass higher than 180;
In one embodiment E is selected from the group of molecules which have a mass higher than 200;
In one embodiment E is selected from the group of molecules which have a mass higher than 220;
In one embodiment E is selected from the group of molecules which have a mass higher than 240;
In one embodiment E is selected from the group of molecules which have a mass higher than 260;
In one embodiment E is selected from the group of molecules which have a mass higher than 280;
In one embodiment E is selected from the group of molecules which have a mass higher than 300;
In one embodiment E is selected from the group of molecules which have a mass higher than 320;
In one embodiment E is selected from the group of molecules which have a mass higher than 340;
In one embodiment E is selected from the group of molecules which have a mass higher than 360;
In one embodiment E is selected from the group of molecules which have a mass higher than 380;
In one embodiment E is selected from the group of molecules which have a mass higher than 400;
In one embodiment E is selected from the group of molecules which have a mass higher than 420;
In one embodiment E is selected from the group of molecules which have a mass higher than 440;
In one embodiment E is selected from the group of molecules which have a mass higher than 460;
In one embodiment E is selected from the group of molecules which have a mass higher than 480;
In one embodiment E is selected from the group of molecules which have a mass higher than 500;
In one embodiment E is selected from the group of molecules which have a mass higher than 550;
In one embodiment E is selected from the group of molecules which have a mass higher than 600;
In one embodiment E is selected from the group of molecules which have a mass higher than 650;
In one embodiment E is selected from the group of molecules which have a mass higher than 700;
In one embodiment E is selected from the group of molecules which have a mass higher than 750;
In one embodiment E is selected from the group of molecules which have a mass higher than 800;
In one embodiment E is selected from the group of molecules which have a mass higher than 850;
In one embodiment E is selected from the group of molecules which have a mass higher than 900;
In one embodiment E is selected from the group of molecules which have a mass higher than 950;
In one embodiment E is selected from the group of molecules which have a mass higher than 1000;
In one embodiment E is selected from the group of molecules which have a mass higher than 1100;
In one embodiment E is selected from the group of molecules which have a mass higher than 1200;
In one embodiment E is selected from the group of molecules which have a mass higher than 1300;
In one embodiment E is selected from the group of molecules which have a mass higher than 1400;
In one embodiment E is selected from the group of molecules which have a mass higher than 1500;
In one embodiment E is selected from the group of molecules which have a mass higher than 1600;
In one embodiment E is selected from the group of molecules which have a mass higher than 1750;
In one embodiment E is selected from the group of molecules which have a mass higher than 2000;
In one embodiment E is selected from the group of molecules which have a mass higher than 2500;
In one embodiment E is selected from the group of molecules which have a mass higher than 3000;
In one embodiment E is selected from the group of molecules which have a mass higher than 4000;
In one embodiment E is selected from the group of molecules which have a mass higher than 5000;
In one embodiment E is selected from the group of molecules which have a mass higher than 7000;
In one embodiment E is selected from the group of molecules which have a mass higher than 10000;
In one embodiment E is selected from the group of molecules which have a mass higher than 15000;
The targeting agent E comprises carbon, hydrogen and possibly heteroatoms.
In one embodiment E is selected from the group of molecules which comprise two heteroatoms;
In one embodiment E is selected from the group of molecules which comprise more than two heteroatoms;
In one embodiment E is selected from the group of molecules which comprise more than three heteroatoms;
In one embodiment E is selected from the group of molecules which comprise more than four heteroatoms;
In one embodiment E is selected from the group of molecules which comprise more than five heteroatoms;
In one embodiment E is selected from the group of molecules which comprise more than six heteroatoms;
In one embodiment E is selected from the group of molecules which comprise more than seven heteroatoms;
In one embodiment E is selected from the group of molecules which comprise more than eight heteroatoms;
In one embodiment E is selected from the group of molecules which comprise more than nine heteroatoms;
In one embodiment E is selected from the group of molecules which comprise more than 10 heteroatoms;
In one embodiment E is selected from the group of molecules which comprise more than 12 heteroatoms;
In one embodiment E is selected from the group of molecules which comprise more than 14 heteroatoms;
In one embodiment E is selected from the group of molecules which comprise more than 16 heteroatoms;
In one embodiment E is selected from the group of molecules which comprise more than 18 heteroatoms;
In one embodiment E is selected from the group of molecules which comprise more than 20 heteroatoms;
In one embodiment E is selected from the group of molecules which comprise more than 25 heteroatoms;
In one embodiment E is selected from the group of molecules which comprise more than 30 heteroatoms;
In one embodiment E is selected from the group of molecules which comprise more than 35 heteroatoms;
In one embodiment E is selected from the group of molecules which comprise more than 40 heteroatoms;
In one embodiment E is selected from the group of molecules which comprise more than 50 heteroatoms;
In one embodiment E is selected from the group of molecules which comprise more than 60 heteroatoms;
In one embodiment E is selected from the group of molecules which comprise more than 80 heteroatoms;
In one embodiment E is selected from the group of molecules which comprise more than 100 heteroatoms;
In one embodiment E is selected from the group of molecules which comprise more than 120 heteroatoms;
In one embodiment E is selected from the group of molecules which comprise more than 150 heteroatoms;
In one embodiment E is selected from the group of molecules which comprise more than 200 heteroatoms;
In one embodiment E is selected from the group of molecules which comprise more than 300 heteroatoms;
The targeting agent E comprises different type of heteroatoms selected but not limited to group comprising oxygen, nitrogen, sulphur, phosphor, seleno, fluoro, chloro, bromo and iodo;
In one embodiment E is selected from the group of molecules which comprise two different type of heteroatoms;
In one embodiment E is selected from the group of molecules which comprise two or more than two different type of heteroatoms;
In one embodiment E is selected from the group of molecules which comprise three or more than three different type of heteroatoms;
In one embodiment E is selected from the group of molecules which comprise four or more than four different type of heteroatoms;
In one embodiment E is selected from the group of molecules which comprise five or more than five different type of heteroatoms;
In one embodiment E is selected from the group of molecules which comprise six or more than six different type of heteroatoms;
In one embodiment E is selected from the group of molecules which comprise seven or more than seven different type of heteroatoms;
The targeting agent E can comprise cyclic structures.
In one embodiment E is selected from the group of molecules which comprise two cyclic structures;
In one embodiment E is selected from the group of molecules which comprise more than two cyclic structures;
In one embodiment E is selected from the group of molecules which comprise more than three cyclic structures;
In one embodiment E is selected from the group of molecules which comprise more than four cyclic structures;
In one embodiment E is selected from the group of molecules which comprise more than five cyclic structures;
In one embodiment E is selected from the group of molecules which comprise more than six cyclic structures;
In one embodiment E is selected from the group of molecules which comprise more than eight cyclic structures;
In one embodiment E is selected from the group of molecules which comprise more than eight cyclic structures;
In one embodiment E is selected from the group of molecules which comprise more than 10 cyclic structures;
In one embodiment E is selected from the group of molecules which comprise more than 15 cyclic structures;
In one embodiment E is selected from the group of molecules which comprise more than 30 cyclic structures;
In one embodiment E is selected from the group of molecules which comprise more than 50 cyclic structures;
The targeting agent E can comprise heteroatoms.
In one embodiment E is selected from the group of molecules which comprise two different type of heteroatoms;
In one embodiment E is selected from the group of molecules which comprise two or more different type of heteroatoms;
In one embodiment E is selected from the group of molecules which comprise three different hetero atoms;
In one embodiment E is selected from the group of molecules which comprise three or more different hetero atoms;
In one embodiment E is selected from the group of molecules which comprise four different hetero atoms;
In one embodiment E is selected from the group of molecules which comprise four or more different hetero atoms;
In one embodiment E is selected from the group of molecules which comprise five different hetero atoms;
In one embodiment E is selected from the group of molecules which comprise five or more different hetero atoms;
In one embodiment E is selected from the group of molecules which comprise six different hetero atoms;
In one embodiment E is selected from the group of molecules which comprise six or more different hetero atoms;
The targeting agent E can comprise aromatic rings;
in one embodiment E is selected from the group of molecules which comprise two or more aromatic rings;
in one embodiment E is selected from the group of molecules which comprise two or more aromatic rings;
in one embodiment E is selected from the group of molecules which comprise more than two aromatic rings;
in one embodiment E is selected from the group of molecules which comprise more than three aromatic rings;
in one embodiment E is selected from the group of molecules which comprise more than four aromatic rings;
in one embodiment E is selected from the group of molecules which comprise more than five aromatic rings;
in one embodiment E is selected from the group of molecules which comprise more than six aromatic rings;
in one embodiment E is selected from the group of molecules which comprise more than eight aromatic rings;
in one embodiment E is selected from the group of molecules which comprise more than eight aromatic rings;
in one embodiment E is selected from the group of molecules which comprise more than 10 aromatic rings;
in one embodiment E is selected from the group of molecules which comprise more than 15 aromatic rings;
in one embodiment E is selected from the group of molecules which comprise more than 20 aromatic rings;
in one embodiment E is selected from the group of molecules which comprise more than 25 aromatic rings;
in one embodiment E is selected from the group of molecules which comprise more than 30 aromatic rings;
in one embodiment E is selected from the group of molecules which comprise more than 40 aromatic rings;
in one embodiment E is selected from the group of molecules which comprise more than 50 aromatic rings;
The targeting agent E comprise heteroatoms.

In one embodiment E is selected from the group of molecules which comprise two different type of heteroatoms, two cyclic structures and a mass higher than 180.

In one embodiment E is selected from the group of molecules which comprise two or more than two different type of heteroatoms, more than one cyclic structures and a mass higher than 250.

In another embodiment E is selected from the group of molecules which comprise two or more than two different type of heteroatoms, two cyclic structures and a mass higher than 180.

In another embodiment E is selected from the group of molecules which comprise two or more than two different type of heteroatoms, three cyclic structures and a mass higher than 300.

The term “bond”, as used herein, is meant to refer to a single, double or triple bond.

The term “linker”, as used herein, is meant to refer to any moiety other than a bond in the above sense, that is capable of covalently connecting two entities within a molecule.

The “fluorination agent” is chemical agent or chemical composition which comprises fluoride anions in free or bound form.

In one embodiment the “fluorination agent” is chemical agent or chemical composition which comprises [¹⁸F]fluoride anions in free or bound form.

In another embodiment the “fluorination agent” is chemical agent or chemical composition which comprises [¹⁹F]fluoride anions in free or bound form.

In a preferred embodiment, the “fluorination agent” comprises a fluorine radioactive isotope derivative.

More preferably the fluorine radioactive isotope derivative is a ¹⁸F derivative. More preferably, the ¹⁸F derivative is 4,7,13,16,21,24-Hexaoxa-1,10-diazabicyclo[8.8.8]-hexacosane K¹⁸F (crownether salt Kryptofix K¹⁸F), K¹⁸F, H¹⁸F, KH¹⁸F₂, Cs¹⁸F, Na¹⁸F or tetraalkylammonium salt of ¹⁸F (e.g. [F-18]tetrabutylammonium fluoride). More preferably, the fluorination agent is K¹⁸F, H¹⁸F, or KH¹⁸F₂, most preferably K¹⁸F (¹⁸F fluoride anion).

The radiofluorination reaction can be carried out, for example in a typical reaction vessel (e.g. Wheaton vial) which is known to someone skilled in the art or in a microreactor. The reaction can be heated by typical methods, e.g. oil bath, heating block or microwave. The radiofluorination reactions are carried out in dimethylformamide with potassium carbonate as base and “kryptofix” as crown-ether. But also other solvents can be used which are well known to experts. These possible conditions include, but are not limited to: dimethylsulfoxid and acetonitril as solvent and tetraalkyl ammonium and tertraalkyl phosphonium carbonate as base. Water and/or alcohol can be involved in such a reaction as co-solvent. The radiofluorination reactions are conducted for one to 60 minutes. Preferred reaction times are five to 50 minutes. Further preferred reaction times are 10 to 40 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). The radiofluorination can be carried out in a “hot-cell” and/or by use of a module (eview: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated or semi-automated synthesis.

The term “active ester moiety” or “active ester”, as used herein, is meant to refer to a carboxylic acid which is activated by a particular substituent in order to ease the conversion of the carboxylic acid with a nucleophile, such as amines. Active esters can be generated in situ or can be isolated in some cases. Examples of acive esters include but are not limited to HOBT-ester, NHS-ester, HOAt-ester, TBTU-ester, OPfP-ester (e.g. Journal of Pharmaceutical Sciences, 58, 2, Pages 281-282), PyBoP-ester, DIC/HOBT-ester, HATU-ester, PyAOP-ester, PyBroP-ester, BroP-ester, mixed anhydride, 1H-imidazol-1-yl-ester (compare e.g. Chan and White (“Fmoc Solid Phase Peptide Synthesis—A Practical Approach”, chapter 7, Oxford University Press or Niemeyer, “Bioconjugation Protocols: Strategies and Methods (Methods in Molecular Biology) (Methods in Molecular Biology)” Humana Press.).

Preferred compounds are:

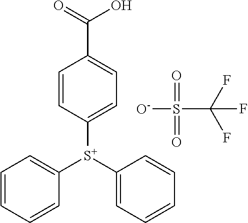

### (4-carboxy-phenyl)-diphenyl-sulfonium trifluoro-methanesulfonate

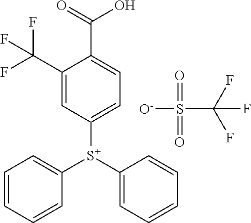

### (4-carboxy-3-trifluoromethyl-phenyl)-diphenyl-sulfonium (trifluoro-methane)sulfonate

In a further aspect the present invention is directed to a composition comprising a compound according to Formula I and a pharmaceutically acceptable carrier or diluent.

In a further aspect the present invention is directed to a kit comprising a sealed vial containing a predetermined quantity of a compound according to Formula I.

In a further aspect the present invention is directed to the use of a compound according to Formula I for the manufacturing of a medicament for treatment of a variety of diseases listed hereafter.

In a further aspect the present invention is directed to a composition comprising a compound according to Formula III and a pharmaceutically acceptable carrier or diluent.

In a further aspect the present invention is directed to a kit comprising a sealed vial containing a predetermined quantity of a compound according to Formula III.

In a further aspect the present invention is directed to the use of a compound according to Formula III for the manufacturing of a medicament for treatment of a variety of diseases listed hereafter.

The term “acid” as employed herein refers to mineral acids, including but not limited to acids such as hydrochloric, hydrobromic, hydroiodic, perchloric, phosphoric, carbonic, nitric or sulphuric acid or to appropriate organic acids which includes but not limited to acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic and sulfanilic acid.

The term “corresponding base of inorganic (or organic) acid” as employed herein refers to base of the so-called “corresponding” acid, including mineral acids but not limited to acids such as carbonic, nitric or sulphuric acid or to appropriate organic acids which includes but not limited to: alkanols ((C₁-C₁₀)alkyl alcohols), acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic and sulfanilic acid.

As used hereinafter in the description of the invention and in the claims, the term “alkoxy (or alkyloxy)” refer to alkyl groups respectively linked by an oxygen atom, with the alkyl portion being as defined above.

The term “[alkyoxyl]-alkyl” refers to a radical of the formula [Ra—O]—Ra— wherein each Ra is an lower alkyl radical as defined above.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl, which themselves can be substituted with one, two or three substituents independently and individually selected from the group comprising halo, nitro, (C₁-C₆)carbonyl, cyano, nitrile, hydroxyl, trifluormethyl, (C₁-C₆)sulfonyl, (C₁-C₆)alkyl, (C₁-C₆)alkoxy and (C₁-C₆)sulfanyl. As outlined above such “aryl” may additionally be substituted by one or several substituents.

The term “heteroaryl” as employed herein refers to groups having 5 to 14 ring atoms; 6, 10 or 14 Π(pi) electrons shared in a cyclic array; and containing carbon atoms (which can be substituted with halo, nitro, (C₁-C₆)carbonyl, cyano, nitrile, trifluormethyl, (C₁-C₆)sulfonyl, (C₁-C₆)alkyl, (C₁-C₆)alkoxy or (C₁-C₆)sulfanyl) and 1, 2, 3 or 4 oxygen, nitrogen or sulfur heteroatoms (where examples of heteroaryl groups are: thienyl, benzo[b]thienyl, naphtho[2,3-b]thienyl, thianthrenyl, furyl, furanyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl and phenoxazinyl groups).

Heteroaryl can be substituted with one, two or three substituents independently and individually selected from the group comprising halo, nitro, (C₁-C₆)carbonyl, cyano, nitrile, hydroxyl, trifluormethyl, (C₁-C₆)sulfonyl, (C₁-C₆)alkyl, (C₁-C₆)alkoxy and (C₁-C₆)sulfanyl. As outlined above such “heteroaryl” may additionally be substituted by one or several substituents.

As used hereinafter in the description of the invention and in the claims, the term “alkyl”, by itself or as part of another group, refers to a straight chain or branched chain alkyl group with 1 to 10 carbon atoms such as, for example methyl, ethyl, propyl, isopropyl, butyl, isobutyl, tert-butyl, pentyl, isopentyl, neopentyl, heptyl, hexyl, decyl. Alkyl groups can also be substituted, such as by halogen atoms, hydroxyl groups, C₁-C₄ alkoxy groups or C₆-C₁₂ aryl groups (which, intern, can also be substituted, such as by 1 to 3 halogen atoms). More preferably alkyl is C₁-C₁₀ alkyl, C₁-C₆ alkyl or C₁-C₄ alkyl.

Whenever the term “substituted” is used, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a pharmaceutical composition. The substituent groups may be selected from halogen atoms, hydroxyl groups, nitro, (C₁-C₆)carbonyl, cyano, nitrile, trifluoromethyl, (C₁-C₆)sulfonyl, (C₁-C₆) alkyl, (C₁-C₆)alkoxy and (C₁-C₆)sulfanyl.

The provision of ¹⁸F anions is known to someone skilled in the art and in one embodiment is achieved by providing aqueous H¹⁸F to which a base, for example in the form of potassium carbonate or tetra alkyl ammonium carbonate is added. The aqueous H¹⁸F may be obtained from a synchrotron.

The term “halo” or “Hal” refers to fluorine (F), chlorine (Cl), bromine (Br), and iodine (I).

Unless otherwise specified, when referring, to the compounds of formula I-V per se as well as to any pharmaceutical composition thereof the present invention includes all of the hydrates, solvates, complexes, and prodrugs of the compounds of the invention. Prodrugs are any covalently bonded compounds, which releases the active parent pharmaceutical according to formula I-V. As outlined above, the compounds of formula I-V may comprise any suitable positron emitting isotope, including ¹⁸F, ¹¹C, ¹⁵C and ¹³N and combinations thereof.

If a chiral center or another form of an isomeric center is present in a compound according to the present invention, all forms of such isomer, including enantiomers and diastereoisomers, are intended to be covered herein. Compounds containing a chiral center may be used as racemic mixture or as an enantiomerically enriched mixture or the racemic mixture may be separated using well-known techniques and an individual enantiomer maybe used alone. In cases in which compounds have unsaturated carbon-carbon bonds double bonds, both the cis-isomer and trans-isomers are within the scope of this invention. In cases wherein compounds may exist in tautomeric forms, such as keto-enol tautomers, each tautomeric form is contemplated as being included within this invention whether existing in equilibrium or predominantly in one form.

Examples of inflammatory and autoimmune diseases are chronic inflammatory intestinal diseases (inflammatory bowel diseases, Crohn's disease, ulcerative colitis), arthritis, atheroma, atherosclerosis, inflammatory cardiomyopathy, pemphigus, asthma, multiple sclerosis, diabetes, type I insulin-dependent diabetes mellitus, rheumatoid arthritis, lupus diseases and other collagenoses, Graves' disease, Hashimoto's disease, “graft-versus-host disease” and transplant rejections. Examples of allergic, infectious and toxin-triggered and ischemia-triggered diseases are: sarcoidosis, asthma, hypersensitive pneumonitis, sepsis, septic shock, endotoxin shock, toxic shock syndrome, toxic liver failure, ARDS (acute respiratory distress syndrome), eclampsia, cachexia, acute viral infections (e.g., mononucleosis, fulminant hepatitis), and post-reperfusion organ damage. An example of a pharmacologically triggered inflammation with pathophysiological relevance is the “first dose response” after administration of anti-T-cell antibodies such as OKT3. An example of systemic inflammation reactions of an origin that is as yet unclear is eclampsia. Examples of neurodegenerative and neuroinflammatory diseases that are associated with a astrocyte activation/MAO regulation are dementia, AIDS dementia, amyotrophic lateral sclerosis, encephalitis, neuropathic pain, Creutzfeldt-Jakob disease, Down's syndrome, diffuse Lewy body disease, Huntington's disease, leukoencephalopathy, encephalopathies, septic encephalopathy, hepatic encephalopathy, multiple sclerosis, Parkinson's disease, Pick's disease, Alzheimer's disease, frontotemporal dementia, hippocampal sclerosis, neurocysticercosis, epilepsy, stroke, ischemia, brain tumors, depression, schizophrenia, drug abuse. The invention, therefore, also relates to the use of imaging compounds for diagnosing these diseases as well as for staging and therapy monitoring.

In a preferred embodiment compounds of this invention are useful for the imaging of multiple sclerosis, Alzheimer's disease, frontotemporal dementia, dementia with Levy bodies, leukoencephalopathy, epilepsy, neuropathic pain, amyotrophic lateral sclerosis, Parkinson's Disease, encephalopathies, brain tumors, depression, drug abuse, chronic inflammatory intestinal diseases, atheroma, atherosclerosis, arthritis, rheumatoid arthritis, pharmacologically triggered inflammation, systemic inflammation of unclear origin.

In a more preferred embodiment compounds of this invention are useful for the imaging of multiple sclerosis, Alzheimer's disease, amyotrophic lateral sclerosis, Parkinson's Disease, leukoencephalopathy, encephalopathies, epilepsy, brain tumors, drug abuse, chronic inflammatory intestinal diseases, atheroma, rheumatoid arthritis, pharmacologically triggered inflammation and systemic inflammation of unclear origin.

In another embodiment the compounds of this invention are useful for the imaging of tissue, in particular tumors and cancers including but not limited to: carcinoma such as bladder, breast, colon, kidney, liver, lung, including small cell lung cancer, esophagus, gall-bladder, ovary, pancreas, stomach, cervix, thyroid, prostate, and skin, hematopoetic tumors of lymphoid and myeloid lineage, tumors of mesenchymal origin, tumors of central peripheral nervous systems, other tumors, including melanoma, seminoma, teratocarcinoma, osteosarcoma, xeroderma pigmentosum, keratoxanthoma, thyroid follicular cancer, and Karposi's sarcoma.

In another embodiment the compounds of this invention are useful for the imaging of cardiovascular diseases including but not limited to:

Hypertonia, peripheral and cardial vascular diseases, coronary diseases, coronary restenosis, such as restenosis after balloon dilation of peripheral vessels, myocardial infarction, acute coronary syndrome, acute coronary syndrome with or without ST-segment elevation, stable and unstable angina pectoris, cardiac insufficiency, prinzmetal's angina, hibernating myocardium, stunned myocardium, tachycardia, atrial tachycardia, arrhythmia, atrial fibrillation, persisting atrial fibrillation, permanent atrial fibrillation, atrial fibrillation with normal or limited left ventricular function, Wolff-Parkinson-White-Syndrome, peripheral impaired circulation, elevated levels of fibrinogen and LDL as well as elevated levels of Plasminogenactivator-Inhibitor 1 (PAI-1), acute coronary syndrome, in particular hypertonia, coronary disease, acute coronary syndrome, angina pectoris, cardiac insufficiency, myocardial infarction and atrial fibrillation.

In accordance with the present invention, the term “cardiac insufficiency” comprises both acute as well as chronical embodiments of such insufficiency, such as more specific or related diseases, such as acute decompensated cardiac insufficiency, right ventricular heart failure, left ventricular heart failure, total cardiac insufficiency, ischemic cardiomyopathy, dilating cardiomyopathy, congenital heart failure, valvular defect, cardiac insufficiency with valvular defects, mitral stenosis, mitral insufficiency, aortic stenosis, aortic insufficiency, tricuspid stenosis, tricuspid insufficiency, pulmonal stenosis, pulmonal insufficiency, combined cardiac valve defect, myocarditis, chronic myocarditis, acute myocarditis, viral myocarditis, diabetic heart insufficiency, alcohol toxic cardiomyopathy, diastolic and systolic heart insufficiency, thromboembolic diseases, post-ischemic reperfusion damages, microvascular and macrovascular damages (vasculitis), arterial and venous thrombosis, oedema, ischemias, such as myocardial infarction, stroke and transitory ischemic attacks. The compounds in accordance with the present invention are furthermore suitable for monitoring coronary artery bypass surgeries (CABG), primary percutaneous transluminal coronary angioplasty (PTCAs), PTCAs after thrombolysis, rescue-PTCA, heart transplantation, open heart surgery, transplantations, bypass surgery, catheter examinations and other surgical operations. Furthermore, the compounds in accordance with the present invention are also useful for imaging of metabolic diseases, such as diabetes, in particular diabetes mellitus, gestational diabetes, insulin-dependent diabetes, non-insulin-dependent diabetes, diabetes-caused diseases, such as retinopathy, nephropathy and neuropathy, metabolic diseases, such as metabolic syndrome, hyperglycemia, hyperinsulinemia, insulin resistance, glucose intolerance, adipositas, arterial sclerosis, dyslipidemia, such as hypercholesterolemia, hypertriglyceridemia, elevated levels of postprandial plasma-triglycerides, hypoalphalipoproteinemia, combined hyperlipidemia, in particular diabetes, metabolic syndrome and dyslipidemia.

As used hereinafter in the description of the invention and in the claims, the term “prodrug” means any covalently bonded compound, which releases the active parent pharmaceutical according to Formula I, preferably the ¹⁸F labelled compound of Formula I.

The term “prodrug” as used throughout this text means the pharmacologically acceptable derivatives such as esters, amides and phosphates, such that the resulting in vivo biotransformation product of the derivative is the active drug as defined in the compounds of Formula (I). The reference by Goodman and Gilman (The Pharmaco-logical Basis of Therapeutics, 8 ed, McGraw-HiM, Int. Ed. 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs of the compounds of the present invention include those compounds wherein for instance a hydroxyl group, such as the hydroxyl group on the asymmetric carbon atom, or an amino group is bonded to any group that, when the prodrug is administered to a patient, cleaves to form a free hydroxyl or free amino, respectively.

Typical examples of prodrugs are described for instance in WO 99/33795, WO 99/33815, WO 99/33793 and WO 99/33792 all incorporated herein by reference.

Prodrugs can be characterized by excellent aqueous solubility, increased bioavailability and are readily metabolized into the active inhibitors in vivo.

Moreover, reference is made to the following examples and example compounds which are given to illustrate not to limit the present invention.

### EXAMPLES

### Experimental Section

### General Procedures:

### 1: Amide Formation for Carboxylic Acid Derivatives and Amine Derivatives Using TBCR as Condensating Agent

To a solution of 1.3 eq. carboxylic acid in DMF (4.3 ml/mmol carboxylic acid) is added 1.3 eq. 4-(4,6-dimethoxy-1,3,5-triazin-2-yl)-4-methylmorpholin-4-ium tetrafluoroborate (TBCR (J. Am. Chem. Soc. 2005, 127, 16912-16920)) and 1.95 eq. N-methyl morpholine. The reaction mixture is stirred for 40 min 1 eq. amine in DMF (1.5 ml/mmol) is added drop by drop. The reaction mixture is stirred between 4 hours to 20 hours. The reaction mixture is reduced by evaporation. A portion of the crude product is dissolved in DMSO and the desired product is purified by preparative HPLC and subsequent lyophilisation of the corresponding HPLC fraction.

3: Fluorination with Radioactive [F-18]Fluoride

Aqueous [¹⁸F]Fluoride (0.1-5 GBq) was trapped on a QMA cartridge and eluted with 5 mg K2.2.2 in 0.95 ml MeCN +1 mg K2CO3 in 50 μl water into a Wheaton vial (5 ml). The solvent is removed by heating at 120° C. for 10 mins under a stream of nitrogen. Anhydrous MeCN (1 ml) is added and evaporated as before. This step is repeated three times. A solution of starting material (1 mg) in 300 μl anhydrous DMF is added. After heating at 120° C. for 10 min the crude reaction mixture is analyzed using analytical HPLC: ACES-C18 50 mm×4.6 mm; solvent gradient: start 5% acetonitril-95% acetonitril in water in 7 min., flow: 2 ml/min. The desired F-18 labeled product is confirmed by co-injection with the corresponding non-radioactive F-19 fluoro-standard on the analytical HPLC. The crude product is pre-purified via a C18 SPE cartridge and (50-2500 MBq) of that pre-purified product are purified by preparative HPLC: ACE 5-C18-HL 250 mm×10 mm; 62% isocratic acetonitril in water 25 min., flow: 3 ml/min The desired product is obtained (30-2000 MBq) as reconfirmed by co-injection with the non-radioactive F-19 fluoro standard on the analytical HPLC. The sample was diluted with 60 ml water and immobilized on a Chromafix C18 (S) cartridge, which was washed with 5 ml water and eluted with 1 ml ethanol to deliver 20-1800 MBq product in 1000 μl EtOH.

### Example 1

### a) Synthesis of (4-carboxyphenyl)(diphenyl)sulfonium trifluoromethanesulfonate (1a)

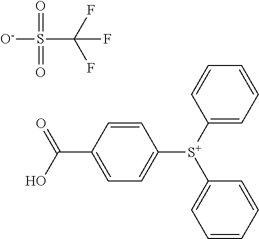

To a solution of 10 g (40.3 mmol) 4-iodo benzoic acid in 150 ml THF was added 1.77 g (44.35 mmol) sodium hydride in one portion. The solution was stirred for 10 min and cooled to −40° C. To this solution was added 59 ml (0.52 mM in THF) (30.83 mmol) diisopropyl magnesium bromide. The temperature was raised to −10° C. within one hour and stirred for another 2.5 h (flask A).

In another flask (flask B) 16.64 g (80.64 mmol) 1,1′-sulfinyldibenzene and 50 ml THF were stirred at −40° C. under inert and dry atmosphere. 14.6 g (80.6 mmol) trimethylsilyl trifluoromethanesulfonate were added drop by drop. The solution in flask B was stirred at −40° C. for 10 min and was added at once to the solution in flask A at −20° C. The mixture was warmed within one hour to −10° C. The reaction mixture was cooled to −70° C. and 100 ml 0.5 M HBr solution was added to the reaction mixture. The mixture was warmed to room temperature and diluted with diethyl ether (300 ml) and 0.5M HBr-solution (200 ml). The organic phase was separated. The aqueous phase was extracted with diethyl ether (1×200 ml) and with dichloromethane (3×200 ml). The combined organic phases were dried and evaporated. The crude product was purified by column chromatography (CH₂Cl₂/MeOH 5:1 - - - >2:1).

MS-ESI: 307 (M⁺, 100).

Elementary analysis:

Calculated: C, 52.63%; H, 3.31%.

Determined: C, 52.65%; H, 3.32%.

### b) {4-[(6-methoxy-1,3-benzothiazol-2-yl)carbamoyl]phenyl}(diphenyl)sulfonium trifluoromethanesulfonate (1b)

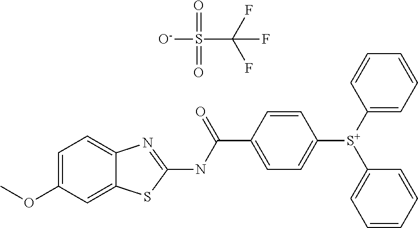

The desired product 1b (25.9 mg) was obtained from 87 mg of 6-methoxy-1,3-benzothiazol-2-amine and 1a according to general procedure 1.

MS-ESI: 469 (M⁺, 100).

Elementary analysis:

| | | | | | | |
|---|---|---|---|---|---|---|
| Calculated: | C | 54.36% | H | 3.42% | N | 4.53% |
| Determined: | C | 54.39% | H | 3.44% | N | 4.52% |

### c) 4-(¹⁸F)fluoro-N-(6-methoxy-1,3-benzothiazol-2-yl)benzamide (1c)

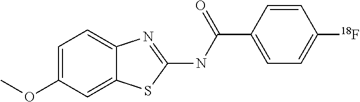

The desired product 1c was obtained from 1b according to the general procedure 3.

### Example 2

An example of the synthesis of compounds of formula I and II and III and IV is depicted in scheme 5:

Para-iodo benzoic acid (4) is converted as magnesium Grignard reagent using iso-propyl magnesium bromide, sodium hydride, diphenyl sulphoxide, O-trimethylsilyl triflate (compare a) A. E. Jensen, W. Dohle, I. Sapountzis, D. M. Lindsay, V. A. Vu, P. Knochel, Synthesis 2002, 565-596; b) S. Imazeki, M. Sumino, K. Fukasawa, M. Ishihara, T. Akiyama, Synthesis 2004, 1648-1654) in THF towards compound 5. The carboxylic acid 5 is coupled with solid phase-bound peptide 6 (trityl resin—These methods are well documented in peptide literature. (Reference: “Fmoc Solid Phase Peptide Synthesis” A practical approach”, Edited by W. C. Chan and P. D. White, Oxford University Press 2000) using the coupling reagent 4-(4,6-Dimethoxy-[1,3,5]triazin-2-yl)-4-methyl-morpholin-4-ium tetrafluoro borate (J. Am. Chem. Soc. 2005, 127, 16912-16920) to obtain solid-phase bound peptide 7. Also other condensating agents are possible. These reagents are well documented in peptide literature. (Reference: ibid). The resin-bound peptide is cleaved by typical acidic methods (e.g. trifluoro acetic acid) so that the peptidic sulphonium derivative 8 with its corresponding base trifluoro acetate as counter ion is liberated.

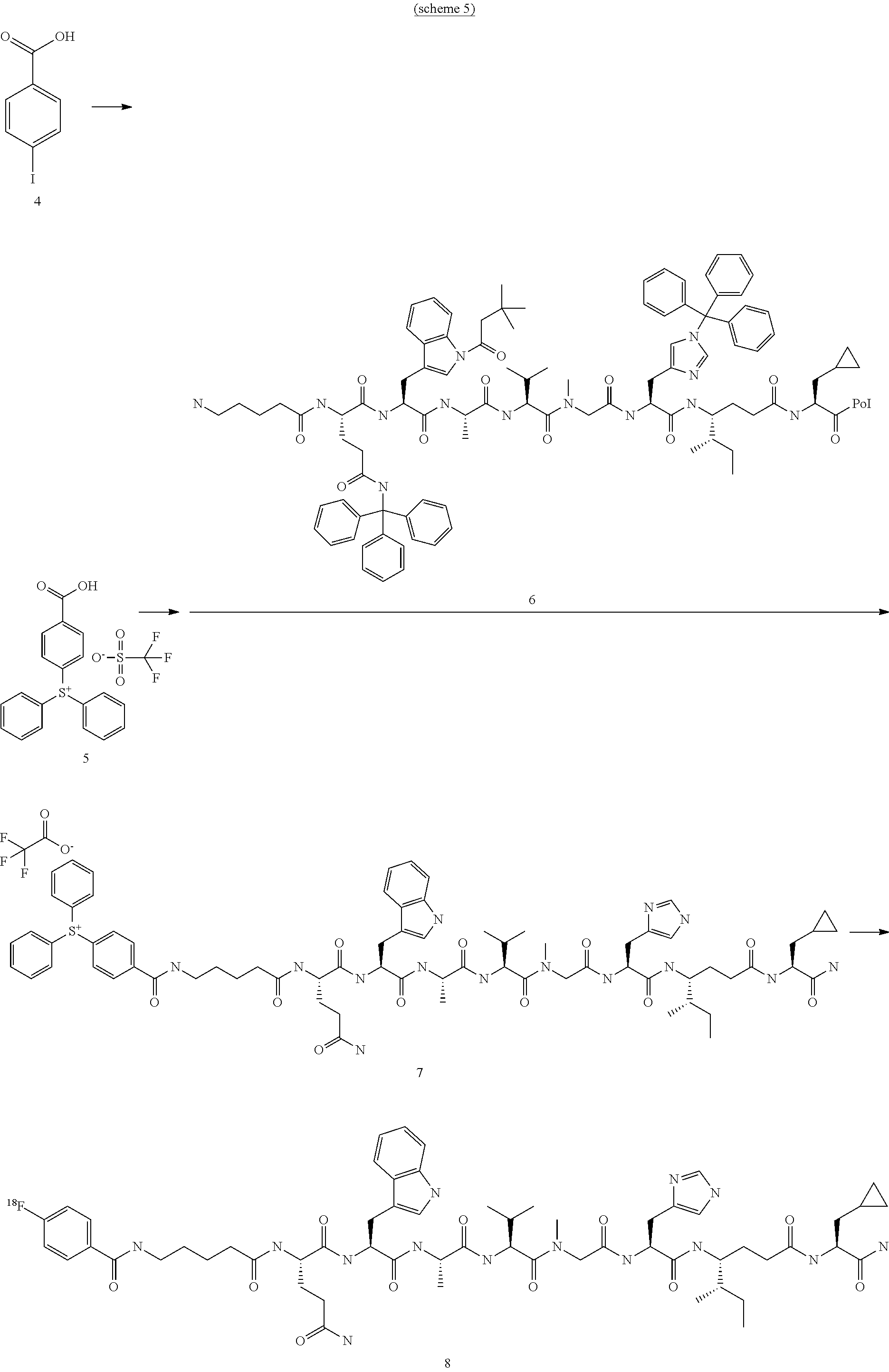

An example of the F-18 labeling of an oligonucleotide is shown in scheme 6. TTA1 (Nucleic Acids Research, 2004, Vol. 32, No. 19, 5757-5765) is equipped with a diphenyl sulfonium derivative (5) by use of a triazine condensating agent (J. Am. Chem. Soc. 2005, 127, 48, 16912-16920). The subsequent F-18 radiolabeling is obtained in reasonable yield, although the specific 10 activity was relatively low due to the fact that the purification of the F-18 labeled compound is achieved under non-optimal circumstances.

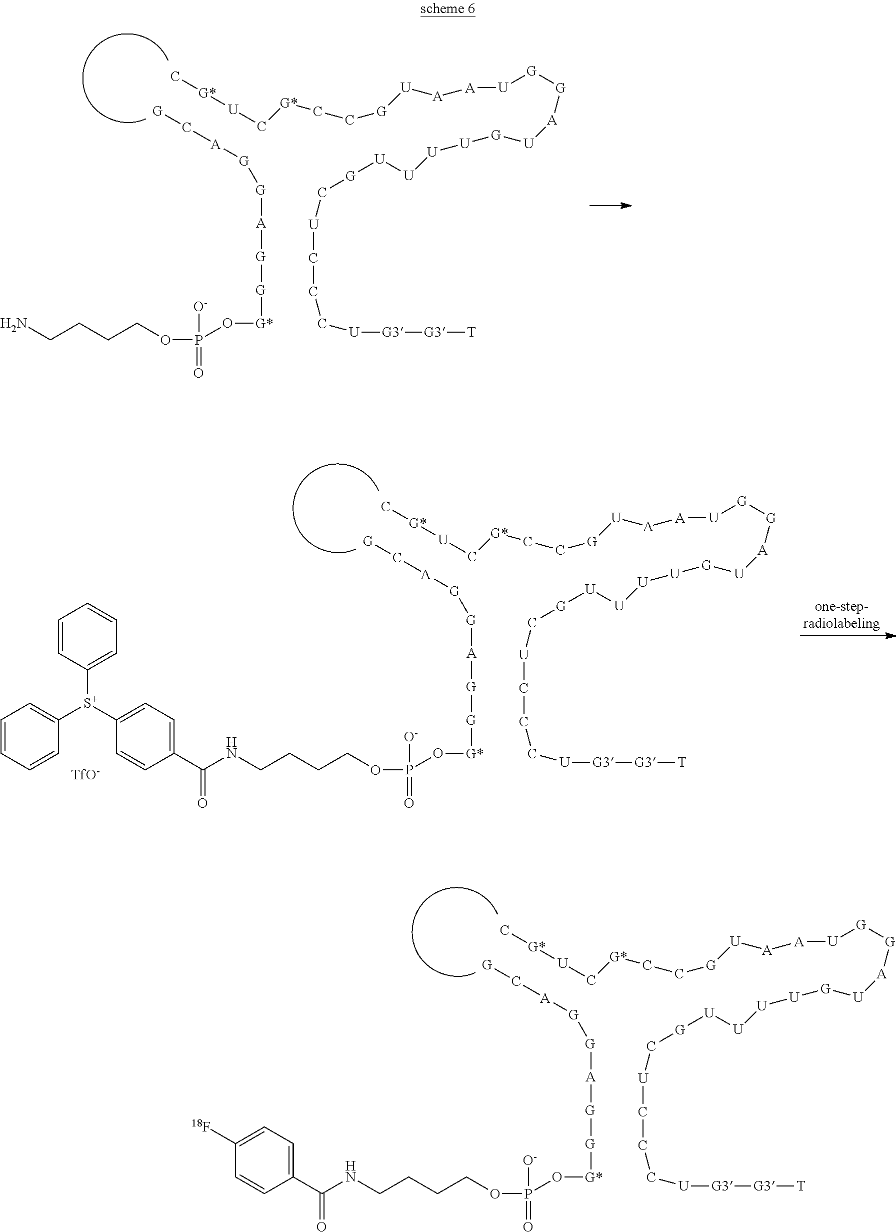

### Example Compounds According to Formula I

| | | |
|---|---|---|
| Compare for corresponding F-18 derivative: Medicinal Chemistry Letters; 15; 21; 2005; 4819-4823; | bis(4-methoxyphenyl)[5- ({4-[4-(2- methoxyphenyl)piperazin- 1- yl]butyl}carbamoyl)pyridin- 2-yl]sulfonium trifluoromethanesulfonate | 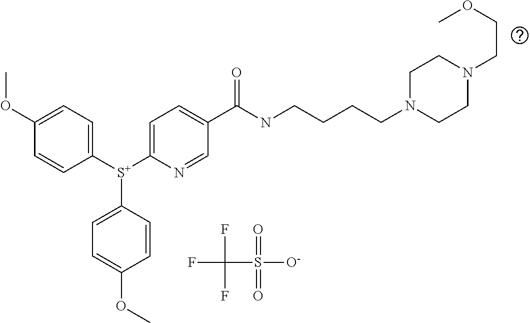 |
| Compare for corresponding F-18 derivative: Journal of Labelled Compounds and Radiopharmaceuticals; 45; 12; 2002; 1045- 1054; | (4-heptanoylphenyl)[bis(4- methylphenyl)]sulfonium trifluoromethanesulfonate | 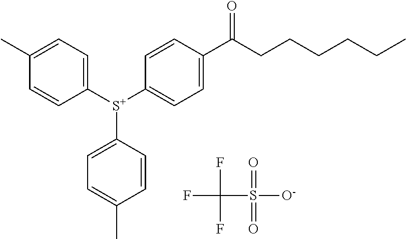 |
| Compare for corresponding F-18 derivative: Journal of Labelled Compounds and Radiopharmaceuticals; 45; 12; 2002; 1045- 1054; | [4-(4- phenoxybulanoyl)phenyl] (diphenyl)sulfonium trifluoromethanesulfonate | 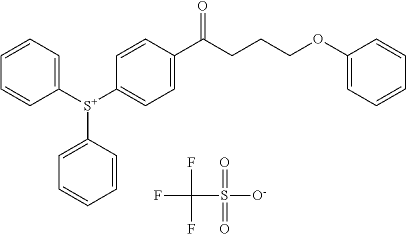 |
| Compare for corresponding F-18 derivative: Journal of Labelled Compounds and Radiopharmaceuticals; 46; 6; 2002; 545-556; | [4-({[5-(2-phenoxyphenyl)- l,3,4-oxadiazol- 2-yl]oxy}carbonyl) phenyl]{bis[4-(propan-2- yl)phenyl]}sulfonium trifluoromethanesulfonate | 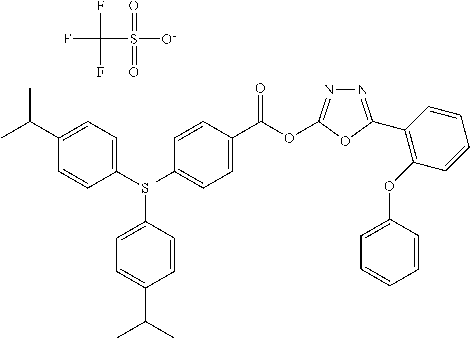 |
| Compare for corresponding F-18 derivative: Journal of Labelled Compounds and Radiopharmaceuticals; 43; 5; 2000; 413-424; | (5-{[(2S)-1-(tert- butoxycarbonyl)azetidin-2- yl]methoxy}pyridin- 2-yl)(diphenyl)sulfonium trifluoromethanesulfonate | 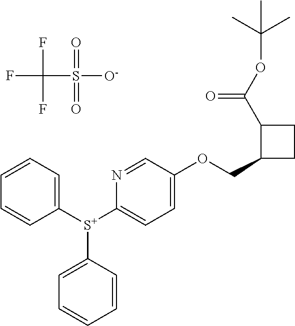 |
| Compare for corresponding F-18 derivative: Journal of Labelled Compounds and Radiopharmaceuticals; 42; 1; 1999; S502-S503; | {5-[(1R,2R,4S)-7-(tert- butoxycarbonyl)-7- azabicyclo[2.2.1]hept- 2-yl]pyridin-2- yl}(diphenyl)sulfonium trifluoromethanesulfonate | 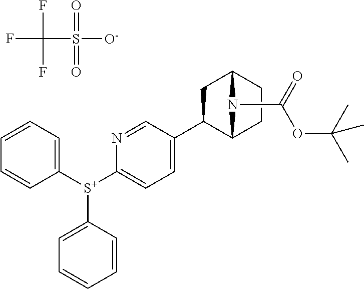 |
| Compare for corresponding F-18 derivative: 6FPWAY Journal of Medicinal Chemistry; 11; 13; 2003; | {6-[(cyclohexylcarbonyl){2- [4-(2- methoxyphenyl)piperazin- 1-yl]ethyl}amino]pyridin-2- yl}[bis(4-methoxyphenyl)] sulfonium trifluoromethanesulfonate | 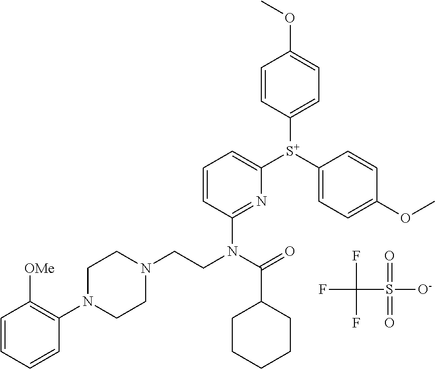 |
| Compare for corresponding F-18 derivative: Journal of Labelled Compounds and Radiopharmaceuticals; 46; 2003; S202-S202; | bis(4-methylphenyl)[4-({1- [2-(4-oxo-2-thioxo-1,4- dihydroquinazolin- 3(2H)-yl)ethyl]piperidin-4- yl}carbonyl)phenyl] sulfonium trifluoromethanesulfonate | 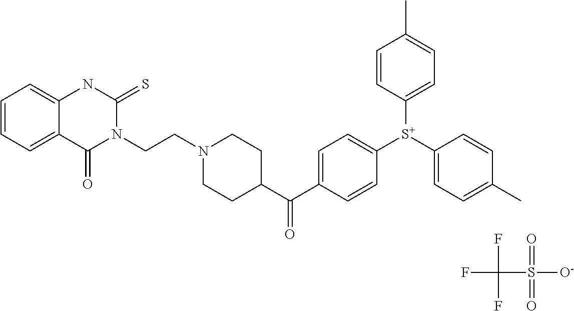 |
| Compare for corresponding F-18 derivative: Journal of Labelled Compounds and Radiopharmaceuticals; 41; 10; 1998; 941-950; | bis(4-methylphenyl)(4-{[1- (4-phenylbutyl)piperidin- 4-yl]carbonyl}phenyl) sulfonium trifluoromethanesulfonate | 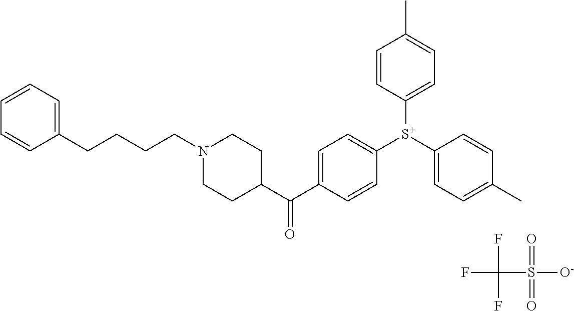 |
| Compare for corresponding F-18 derivative: Chemistry Letters; English; 9; 1995; 835- 836; | [4-({l-[2-(2-methyl-4-oxo- 4H-pyrido[1,2-a]pyrimidin- 3-yl)ethyl]piperidin-4- yl}carbonyl)phenyl][bis(4- methylphenyl)]sulfonium trifluoromethanesulfonate | 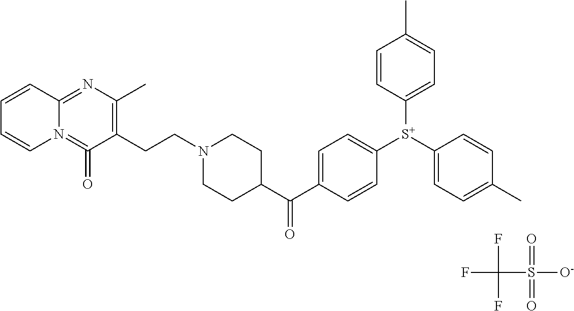 |
| Compare for corresponding F-18 derivative: Journal of Medicinal Chemistry; 50; 16; 2007; 3814-3824; | {5′-(1R,2R,4S)-7-methyl-7- azabicyclo[2.2.1]hept-2-yl]- 3,3'- bipyridin-6-yl}[bis(4- methylphenyl)]sulfonium trifluoromethanesulfonate | 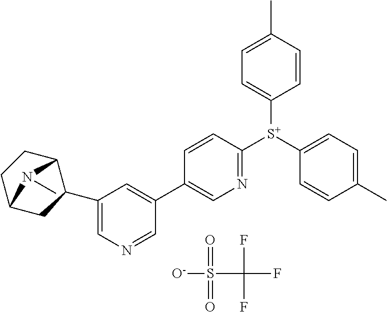 |
| Compare for corresponding F-18 derivative: Journal of Medicinal Chemistry; English; 50; 3; 2007; 489-500; | {6-[4-({4-[4-(2- methoxyphenyl) piperazin-1- yl]butyl}carbamoyl) phenyl]pyridin-2-yl}[bis(4- methylphenyl)]sulfonium trifluoromethanesulfonate | 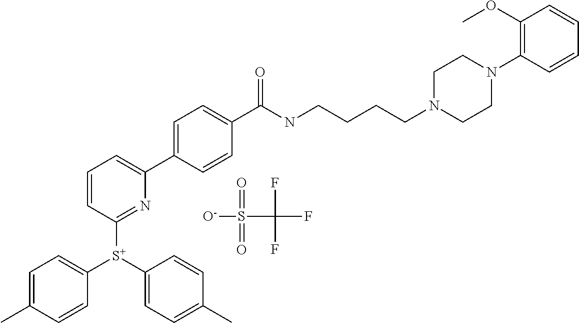 |
| Compare for corresponding F-18 derivative: Tetrahedron Letters; 48; 49; 2007; 8632- 8635. | {3-[acetyl(2,5- dimethoxybenzyl)amino]-4- phenoxyphenyl}(diphenyl) sulfonium trifluoromethanesulfonate | 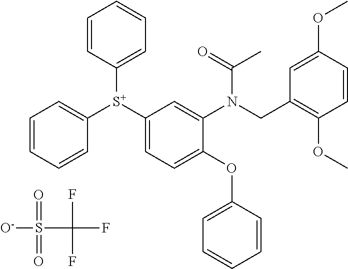 |
| Compare for corresponding F-18 derivative: WO2007/20400 Al | {5-[6-(ethoxymethoxy)-1,3- benzothiazol-2-yl]-2- [formyl(methyl) amino]phenyl}(diphenyl) sulfonium trifluoromethanesulfonate | 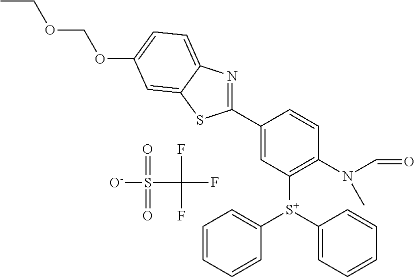 |
| Compare for corresponding F-18 derivative: WO2007/20400 A1 | {2-[(tert- butoxycarbonyl)(methyl) amino]-5-[6- (ethoxymethoxy)- 1,3-benzothiazol-2- yl]phenyl}[bis(4- methoxyphenyl)]sulfonium 4-methylbenzenesulfonate | 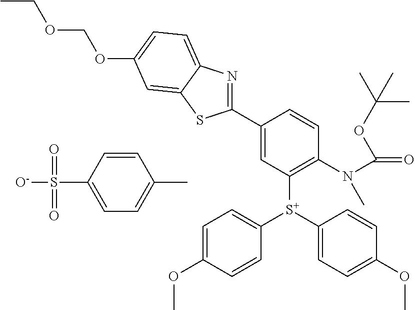 |

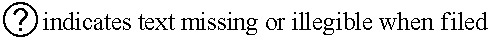

The features of the present invention disclosed in the specification, the claims and/or in the accompanying drawings, may, both separately and in any combination thereof, be material for realizing the invention in various forms thereof.

### Experimental Part for Peptide Synthesis

General
Peptide synthesis was carried out using Rink-Amide resin (0.68 mmol/g) following standard Fmoc strategy (Fields G B, Noble R L. Solid phase peptide synthesis utilizing 9-fluorenylmethoxycarbonyl amino acids. Int. J. Pept. Protein Res. 1990; 35: 161-214). All amino acid residues are, if not further specified, L-amino acid residues.

Fmoc-Deprotection (General Procedure)
The resin-bound Fmoc peptide was treated with 20% piperidine in DMF (v/v) for 5 min and a second time for 20 min. The resin was washed with DMF (2×), CH₂Cl₂ (2×), and DMF (2×).

HBTU/HOBT Coupling (General Procedure)
A solution of Fmoc-Xaa-OH (4 eq), HBTU (4 eq), HOBT (4 eq), DIEA (4 eq) in DMF was added to the resin-bound free amine peptide and shaken for 90 min at room temperature. The coupling was repeated for another 60 min and the resin was washed with DMF (2×), CH₂Cl₂ (2×), and DMF (2×).

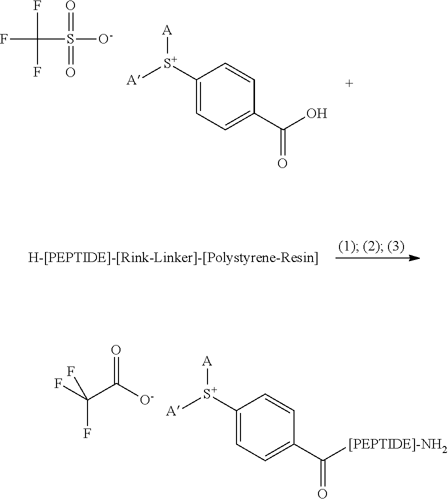

Scheme Xprmtl1 Examples of the synthesis of compounds according to formula I: (1) coupling; (2) cleavage from resin; (3) HPLC-purification; A=A′=phenyl or 4-toluoyl

Coupling Procedure According to Scheme Xprmtl1 (General Procedure A)
A solution of (4-carboxyphenyl)-diaryl-sulfonium trifluoromethanesulfonate (2 eq), HBTU (2 eq), HOBT (2 eq), DIEA (4 eq) in DMF was added to the resin-bound free amine peptide and shaken for 4 h at ambient temperature. The resin was washed with DMF (4×) and CH₂Cl₂ (4×) and dried in vacuum.

Coupling Procedure According to Scheme Xprmtl1 (General Procedure B)
A solution of (4-carboxyphenyl)-diaryl-sulfonium trifluoromethanesulfonate (2 eq), 4-(4,6-Dimethoxy-[1,3,5]triazin-2-yl)-4-methyl-morpholin-4-ium tetrafluoroborate (2 eq), N-methyl-morpholine (2 eq) in DMF was added to the resin-bound free amine peptide and shaken for 4 h at ambient temperature. The resin was washed with DMF (4×) and CH₂Cl₂ (4×) and dried in vacuum.

Cleavage from Resin (According to Scheme Xprmtl1)
The peptide was cleaved from resin by treatment with a mixture of TFA, water, phenol and triisopropylsilane (85:5:5:5 v-%). The peptide was then precipitated with methyl-tert-butyl ether, the solvent was removed by centrifugation, and the crude product was dried in vacuum.

HPLC-Purification (According to Scheme Xprmtl1)
Column material and size: Luna C-18; 5μ; 150×21.2 mm
Mobile Phase: A) water+0.1% TFA
   B) acetonitrile+0.1% TFA
Gradient: 5-40% B, 20 min
Fractions having the correct molecular weight (analytical LC-ESI-MS) were collected and lyophilized to give the pure product.

### Product Examples

TABLE
products and MS-analytical results

| | | | PEPTIDE | | |
|---|---|---|---|---|---|
| | | | (according to | | |
| | | Coupling | scheme | ESI-MS | ESI-MS |
| Entry | A, A′ | procedure | Xprmtl1) | [M]⁺; calc. | (found) |
| 1 | phenyl | A | Ala(SO₃H)- | 1488.7 | 1489.1 |
| | | | Ava-Gln-Trp- | | |
| | | | Ala-Val-Gly- | | |
| | | | His-[4-R- | | |
| | | | amino-5-S- | | |
| | | | methylheptanoyl]- | | |
| | | | Leu | | |
| 2 | phenyl | B | Ava-Gln-Trp- | 1337.7 | 1338.0 |
| | | | Ala-Val-Gly- | | |
| | | | His-[4-R- | | |
| | | | amino-5-S- | | |
| | | | methylheptanoyl]- | | |
| | | | Leu | | |
| 3 | 4-toluyl | B | Ava-Gln-Trp- | 1377.7 | 1378.0 |
| | | | Ala-Val- | | |
| | | | NMeGly-His- | | |
| | | | [4-R-amino- | | |
| | | | 5-S- | | |
| | | | methylheptanoyl]- | | |
| | | | Cpa | | |
| 4 | phenyl | B | Gly-Tyr-Ala- | 734.3 | 734.6 |
| | | | His | | |
| 5 | phenyl | B | Ava-Gln-Trp- | 1349.7 | 1349.8 |
| | | | Ala-Val- | | |
| | | | NMeGly-His- | | |
| | | | [4-R-amino- | | |
| | | | 5-S- | | |
| | | | methylheptanoyl]- | | |
| | | | Cpa | | |
| 6 | phenyl | B | Val-βAla- | 680.3 | 680.2 |
| | | | Phe-Gly | | |

HPLC Analysis of Purified Products
Table entry 1

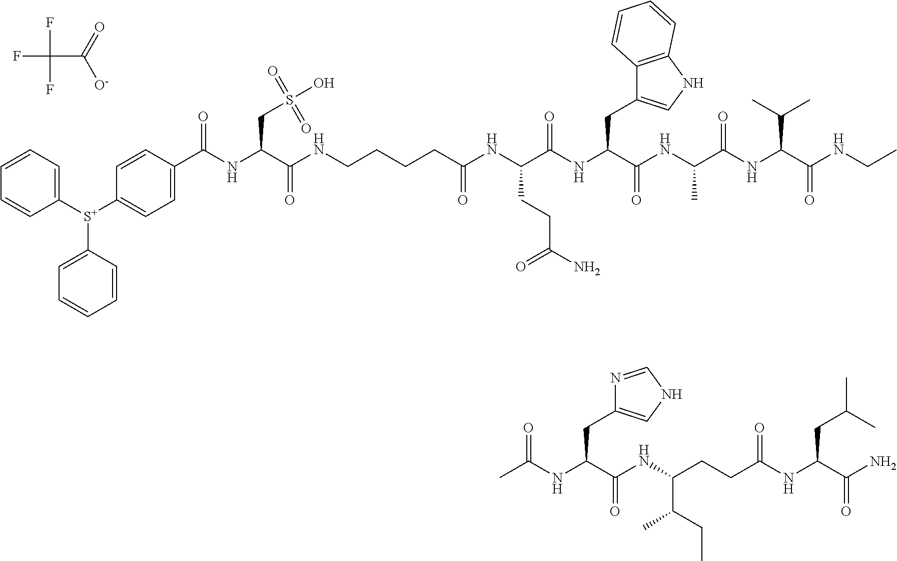

### N²-{5-[(N-{[4-(diphenylsulfonio)phenyl]carbonyl}-3-sulfo-L-alanyl)amino]pentanoyl}-L-glutaminyl-L-tryptophyl-L-alanyl-L-valylglycyl-N-[(4R,5S)-1-{[(2S)-1-amino-4-methyl-1-oxopentan-2-yl]amino}-5-methyl-1-oxoheptan-4-yl]-L-histidinamide trifluoroacetate

Figure 1
The peaks (1)+(2) are system peaks; The peak 3 correspond to the product.
   Table Entry 2

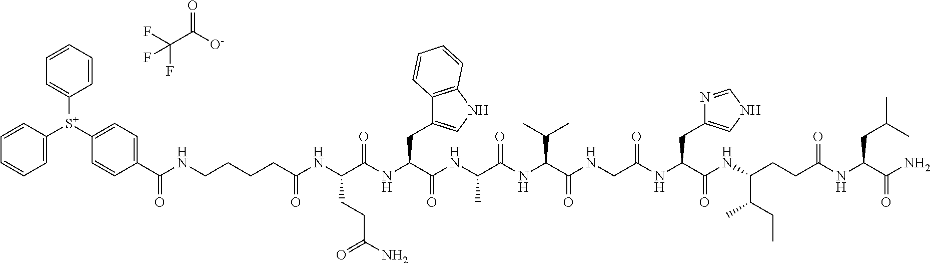

### N²-[5-({[4-(diphenylsulfonio)phenyl]carbonyl}amino)pentanoyl]-L-glutaminyl-L-tryptophyl-L-alanyl-L-valylglycyl-N-[(4R,5S)-1-{[(2S)-1-amino-4-methyl-1-oxopentan-2-yl]amino}-5-methyl-1-oxoheptan-4-yl]-L-histidinamide trifluoroacetate

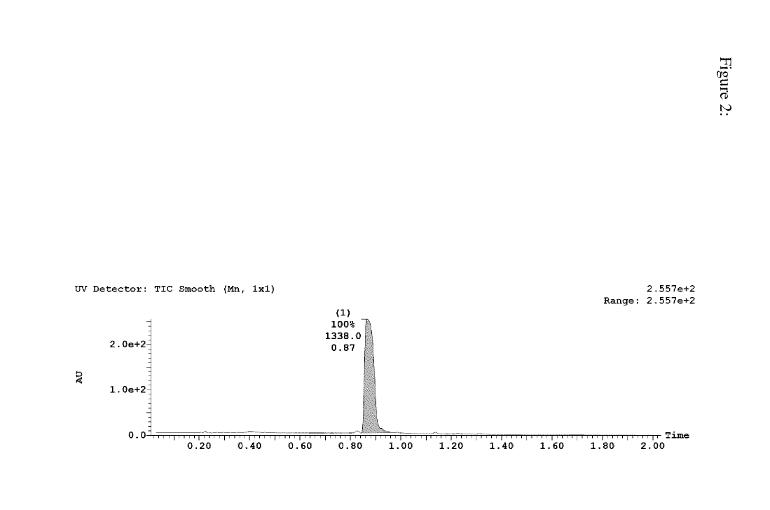

Figure 2
The peak (1) corresponds to the product.
   Table entry 3

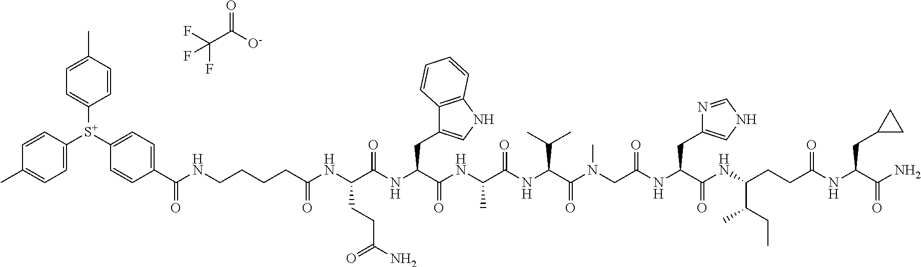

### N²-{5-[({4-[bis(4-methylphenyl)sulfonio]phenyl}carbonyl)amino]pentanoyl}-L-glutaminyl-L-tryptophyl-L-alanyl-L-valyl-N-methylglycyl-N-[(4R,5S)-1-{[2S)-1-amino-3-cyclopropyl-1-oxopropan-2-yl]amino}-5-methyl-1-oxoheptan-4-yl]-L-histidinamide trifluoroacetate

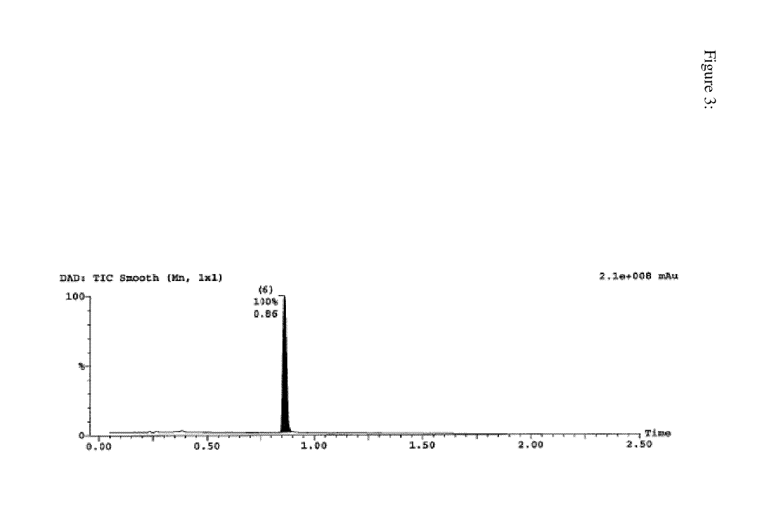

Figure 3
The peak (6) corresponds to the product.
Table entry 4

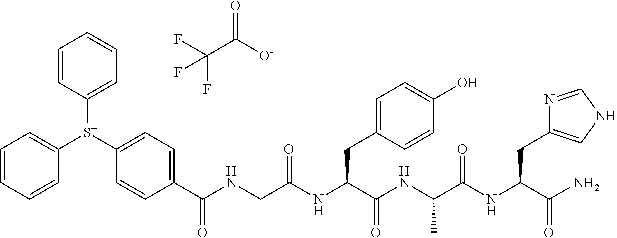

### N-{[4-(diphenylsulfonio)phenyl]carbonyl}glycyl-L-tyrosyl-L-alanyl-L-histidinamide trifluoroacetate

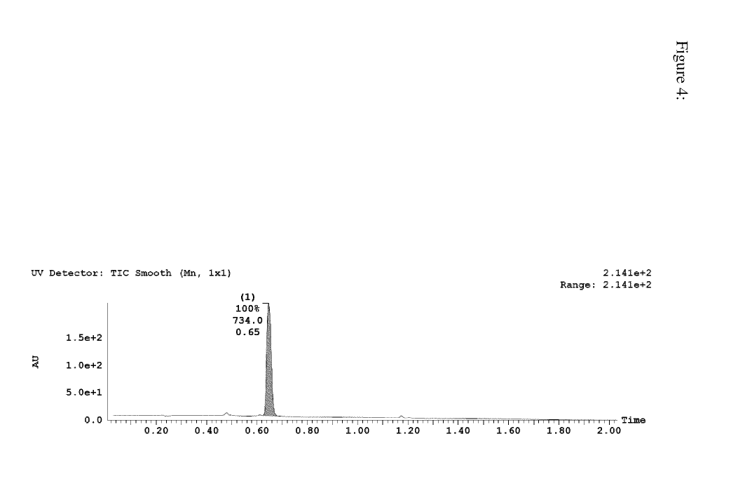

Figure 4
The peak (1) corresponds to the product.
Table entry 5

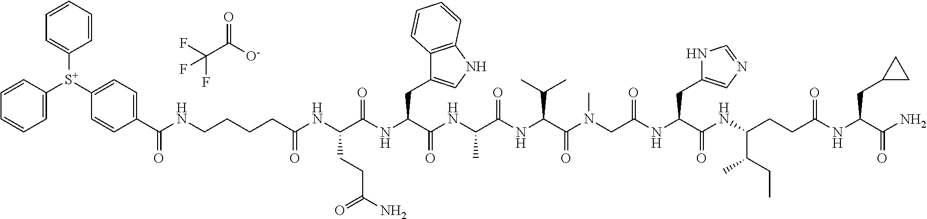

### N²-[5-({[4-(diphenylsulfonio)phenyl]carbonyl}amino)pentanoyl]-L-glutaminyl-L-tryptophyl-L-alanyl-L-valyl-N-methylglycyl-N-[(4R,5S)-1-{[(2S)-1-amino-3-cyclopropyl-1-oxopropan-2-yl]amino}-5-methyl-1-oxoheptan-4-yl]-L-histidinamide trifluoroacetate

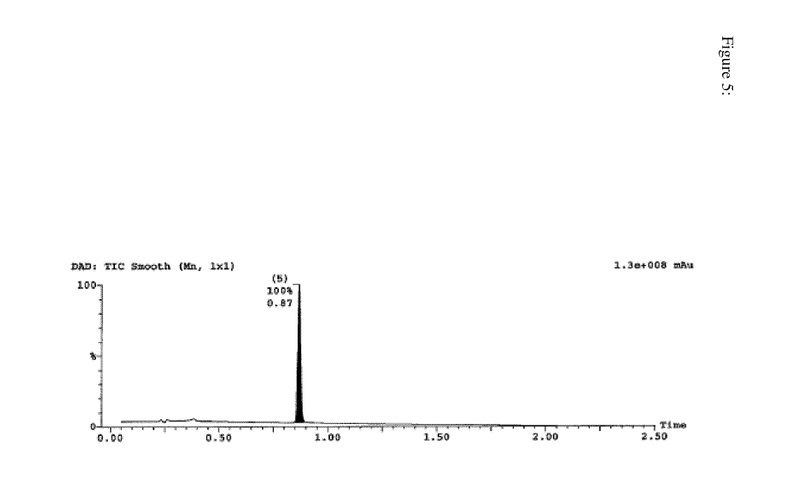

Figure 5
The peak (5) corresponds to the product.
Table entry 6

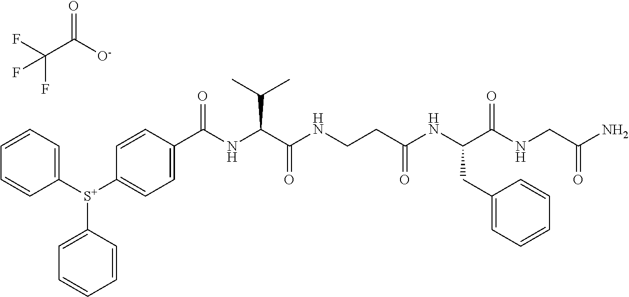

### N-{[4-(diphenylsulfonio)phenyl]carbonyl}-L-valyl-beta-alanyl-L-phenylalanylglycinamide trifluoroacetate

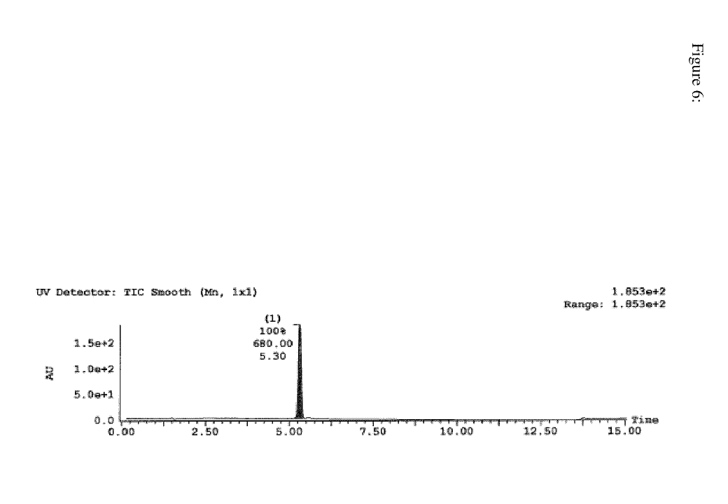

Figure 6
The peak (1) corresponds to the product.

1. A compound of Formula I 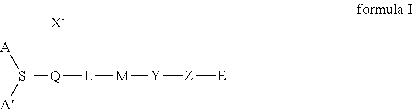
wherein A, A′ and Q are independently at each occurrence and individually selected from the group comprising aryl, substituted aryl, heteroaryl and substituted heteroaryl,
wherein optionally either A and A′ A and Q or A′ and Q are linked to each other via R⁴,
wherein, when substituted, there is one substituent or several substituents on said aryl or said heteroaryl, and the one or several substituent(s) is (are) located at any position of said aryl or heteroaryl,
wherein S is sulfur,
wherein X⁻ is selected from the group comprising a corresponding base of an inorganic acid and a corresponding base of an organic acid,
wherein L-M-Y—Z is selected from the group comprising a bond and a linker, said bond being selected from a single, double or triple bond, said bond connecting E to Q, said linker connecting E to Q,
wherein E is a targeting agent,
R⁴ being selected from the group comprising: bond, oxygen atom, sulphur atom, (N-alkyl) nitrogen, in particular (N—(C₁-C₄)alkyl)nitrogen, (C₁-C₃)alkylene, and (C₂-C₃)alkylene, and
including all isomeric forms of said compound, including but not limited to enantiomers and diastereoisomers as well as racemic mixtures;
and any pharmaceutically acceptable salt, ester, amide, complex or prodrug thereof.

2. A method for obtaining compounds of Formula II
F-Q²-L-M-Y—Z-E II
wherein A, A′ and Q² are independently at each occurrence and individually selected from the group comprising aryl, substituted aryl, heteroaryl and substituted heteroaryl, wherein optionally either A and A′, A and Q² or A¹ and Q² are linked to each other via R⁴,
wherein, when substituted, there is one substituent or several substituents on said aryl or said heteroaryl, and the one or several substituent(s) is (are) located at any position of said aryl or heteroaryl,
wherein S is sulfur,
wherein X⁻ is selected from the group comprising a corresponding base of an inorganic acid and a corresponding base of an organic acid,
wherein L-M-Y—Z is selected from the group comprising a bond and a linker, said bond being selected from a single, double or triple bond, said bond connecting E to Q², said linker connecting E to Q²,
wherein E is a targeting agent, and
R⁴ being selected from the group comprising: bond, oxygen atom, sulphur atom, (N-alkyl) nitrogen, in particular (N—(C₁-C₄)alkyl)nitrogen, (C₁-C₃)alkylene, and (C₂-C₃)alkylene, and
Q² is Q
comprising the step
Reacting compound of Formula I with fluorination agent.

3. A compound according to formula III 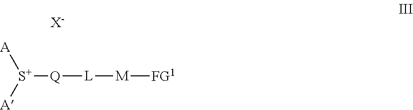
wherein A, A′, S, Q, L, X⁻ and M are as defined above, and
wherein FG¹ is selected from the group comprising
a) hydroxy,
b) iodo,
c) bromo,
d) chloro,
e) N₃,
f) C≡CH,
g) C(O)OR³,
h) active ester moiety,
i) C(O)-Hal,
j) NHR¹,
k) N═C═O,
l) N═C═S,
m) O—S(O)₂-aryl,
n) OS(O)₂-alkyl,
o) SO₂-Hal,
p) S₃H,
q) SH,
r) O—C(═O)-Hal,
s) O—C(═S)-Hal,
t) 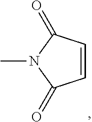
u) 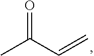
v) 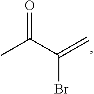
w) 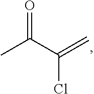
x) 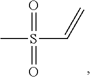
y) 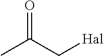
and
z) 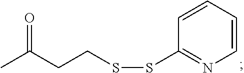
wherein R¹ is as defined above, and wherein R³ is selected from the group comprising
a) hydrogen,
b) active ester moiety,
c) (C₁-C₆)alkyl,
d) (C₂-C₆)alkenyl and
e) aryl alkyl; and
wherein Hal is a halogen, such as F, Cl, Br or I
including all isomeric forms of said compound, including but not limited to enantiomers and diastereoisomers as well as racemic mixtures;
and any pharmaceutically acceptable salt, ester, amide, complex or prodrug thereof.

4. A method for obtaining compounds of Formula IV
¹⁸F-Q²-L-M-FG¹ IV
wherein
Q² is Q;
L is selected from the group comprising
a) —C(═O)H,
b) —S(═O)₂H,
c) —S(═O)H,
d) —N(H)—C(═O)H and
e) —C≡C—C(═O)—,
wherein L is in ortho, meta, para or any other position to S⁺; and
M is selected from the group comprising
a) bond, said bond being selected from a single, double or triple bond,
b) —(CH₂)_{d}—,
c) —(CH₂)_{d}-D-(CH₂)_{d}—,
d) —N(R¹)—(CH₂)_{d}— and
e) —N(R¹)—(CH₂)ₚ—(CH₂—O—CH₂)ₖ—(CH₂)ₚ—;
FG¹ is selected from the group comprising
a) hydroxy,
b) iodo,
c) bromo,
d) chloro,
e) N₃,
f) C≡CH,
g) C(O)OR³,
h) active ester moiety,
i) C(O)-Hal,
j) NHR¹,
k) N═C═O,
l) N═C═S,
m) O—S(O)₂-aryl,
n) OS(O)₂-alkyl,
o) SO₂-Hal,
p) S₃H,
q) SH,
r) O—C(═O)-Hal,
s) O—C(═S)-Hal,
t) 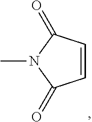
u) 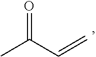
v) 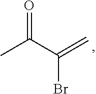
w) 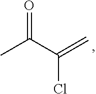
x) 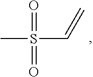
y) 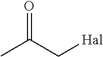
and
z) 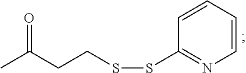
comprising the step
Reacting compound of Formula III with fluorination agent.

5. A method of preparing a compound according to formula I according to claim 1,
comprising the step
Reacting a compound according to formula III with a compound according to formula V
E-FG² V
wherein FG² is identical to FG¹ and
E is a targeting agent.

6. A compound according to claim 1 wherein E is a peptide (or a peptidomimetic) or an oligonucleotide or a small molecule,

7. A pharmaceutical composition comprising a compound according to formula I or III and a pharmaceutically acceptable carrier or diluent.

8. A kit comprising a sealed vial containing a predetermined quantity of a compound according to formula I according to claim 1 and/or a compound according to formula III 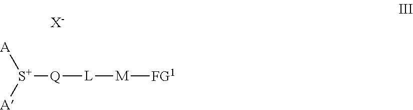
wherein A, A′, S, Q, L, X⁻ and M are as defined above, and
wherein FG¹ is selected from the group comprising
f) hydroxy,
g) iodo,
h) bromo,
i) chloro,
j) N₃,
aa) C≡CH,
bb) C(O)OR³,
cc) active ester moiety,
dd) C(O)-Hal,
ee) NHR¹,
ff) N═C═O,
gg) N═C═S,
hh) O—S(O)₂-aryl,
ii) OS(O)₂-alkyl,
jj) SO₂-Hal,
kk) S₃H,
ll) SH,
mm) O—C(═O)-Hal,
nn) O—C(═S)-Hal,
oo) 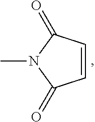
pp) 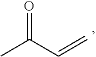
qq) 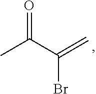
rr) 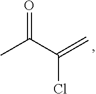
ss) 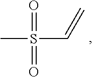
tt) 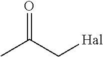
and
uu) 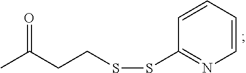
wherein R¹ is as defined above, and wherein R³ is selected from the group comprising
a) hydrogen,
b) active ester moiety,
c) (C₁-C₆)alkyl,
d) (C₂-C₆)alkenyl and
e) aryl alkyl; and
wherein Hal is a halogen, such as F, Cl, Br or I
in one sealed vial or in separate sealed vial.

9. (canceled)

10. (canceled)

11. A method of treating CNS diseases comprising administering an effective amount of a compound according to formula I of claim 1.

12. A method of using a compound of Formula II of claim 2 as an imaging agent comprising administering a compound of Formula II to a patient and imaging the patient.

13. A method of treating CNS diseases comprising administering an effective amount of a compound according to formula II of claim 2.

14. A method of treating CNS diseases comprising administering an effective amount of a compound according to formula III of claim 3.

15. A method of treating CNS diseases comprising administering an effective amount of a compound according to formula IV of claims 4.

16. A method of using a compound of Formula IV of claim 4 as an imaging agent comprising administering a compound of Formula IV to a patient and taking an image of said patient.
